(19) United States
(12) Patent Application Publication
(10) Pub. No.: US 2025/0049729 A1
(43) Pub. Date: Feb. 13, 2025

(54) POEGMA-BASED LIPID NANOPARTICLES

(71) Applicant: Duke University, Durham, NC (US)

(72) Inventors: Ashutosh Chilkoti, Durham, NC (US); Soumen Saha, Durham, NC (US); Parul Sirohi, Durham, NC (US); Angus Hucknall, Durham, NC (US); Sonal Deshpande, Durham, NC (US)

(21) Appl. No.: 18/704,387

(22) PCT Filed: Oct. 25, 2022
(86) PCT No.: PCT/US2022/078659

§ 371 Date: Apr. 24, 2024

Related U.S. Application Data
(60) Provisional application No. 63/271,595, filed on Oct. 25, 2021.

(51) Int. Cl. A61K 9/51; A61K 31/7105; A61K 31/711; A61K 39/215

(52) U.S. Cl. CPC A61K 9/5146; A61K 9/5123; A61K 31/7105; A61K 31/711; A61K 39/215

(57) ABSTRACT

Disclosed herein are lipid nanoparticles including a POEGMA-lipid conjugate that can effectively encapsulate and deliver therapeutics without the immune consequences suffered by PEG-based counterparts. An example lipid nanoparticle includes an ionizable lipid, a phospholipid, a sterol, a POEGMA-lipid conjugate, and a therapeutic. Also disclosed herein are pharmaceutical compositions that include the POEGMA-based lipid nanoparticles, methods of treating a disease or disorder, and methods of delivering a therapeutic to a cell.

### CROSS-REFERENCE TO RELATED APPLICATIONS

This application claims priority to U.S. Provisional Patent Application No. 63/271,595 filed on Oct. 25, 2021, which is incorporated fully herein by reference.

### REFERENCE TO AN ELECTRONIC SEQUENCE LISTING

The contents of the electronic sequence listing (Name: 028193-9376-WO01_Sequence_Listing.xml; Size: 9,210 bytes; Date of Creation: Oct. 25, 2022) is herein incorporated by reference in its entirety.

### TECHNICAL FIELD

The present disclosure relates to lipid nanoparticles that include POEGMA-lipid conjugates and their use in biomedical applications, such as drug delivery.

### INTRODUCTION

Lipid nanoparticles (LNPs) have emerged as promising carriers for nucleic acid delivery (e.g., mRNA) owing to their biocompatibility, efficient complexation with the payload, cellular uptake, and successful endosomal escape. LNPs have been in the spotlight as an important component of the COVID-19 mRNA vaccines because of their role in effectively protecting and transporting mRNA to cells. LNPs typically include a PEGylated lipid that can provide stealth properties to the LNPs. LNPs with PEG coating have much longer plasma half-life than native LNPs due to reduced opsonization and improved solubility. Unfortunately, in vivo administration of PEGylated LNPs has several limitations including immunogenicity, allergic side reaction, and enhanced clearance by induced and pre-existing PEG antibodies. Repeated administration of PEG can also form vacuoles in major organs due to its non-biodegradable structure and clearance by the RES. Moreover, PEG intolerance has caused the early termination of several clinical trials and the withdrawal of several therapeutics from the market. As per the CDC, PEG has been identified as one of the major components responsible for allergic reactions to LNP-based Pfizer-BioNTech covid vaccine.

### SUMMARY

In one aspect, disclosed are lipid nanoparticles including an ionizable lipid; a phospholipid; a sterol, a poly[oligo (ethylene glycol) ether methacrylate] (POEGMA)-lipid conjugate at less than 10 mol %, wherein the POEGMA has a number average molecular weight of less than 100 kDa; and a therapeutic.

In another aspect, disclosed are lipid nanoparticles including (heptadecan-9-yl 8-((2-hydroxyethyl)(6-oxO-6-(undecyloxy) hexyl) amino) octanoate) (SM-102); DSPC; cholesterol; a POEGMA-lipid conjugate at about 0.25 mol % to about 3 mol %, wherein the POEGMA has a number average molecular weight of about 1 kDa to about 50 kDa; and an mRNA.

In another aspect, disclosed are pharmaceutical compositions including one or more lipid nanoparticles as disclosed herein; and a pharmaceutically acceptable excipient.

In another aspect, disclosed are methods of treating a disease or a disorder in a subject in need thereof, the method including administering to the subject an effective amount of one or more lipid nanoparticles as disclosed herein, optionally in combination with a pharmaceutically acceptable excipient.

In another aspect, disclosed are methods of delivering a therapeutic to a cell, the method including contacting the cell with one or more lipid nanoparticles as disclosed herein, whereby the therapeutic is delivered to the cell.

### BRIEF DESCRIPTION OF THE DRAWINGS

FIG. 1 shows gel permeation chromatography-multi-angle light scattering (GPC-MALS) (FIG. 1A) and reverse phase high performance liquid chromatography (HPLC) (FIG. 1B) trace of example azido poly[oligo (ethylene glycol) ether methacrylate] (POEGMA).

FIG. 2 shows purification and characterization of example POEGMA-lipid conjugates (POEGMAL). FIG. 2A: schematic of purification of POEGMAL. FIG. 2B: Typical TLC trace of a POEGMAlyted lipid. FIG. 2C: physical appearance of POEGMA₁₀ and POEGMAL₁₀. FIG. 2D: Ratios of theoretical number of protons between δ 0.5-2.5 and δ 7.3-7.8 and the corresponding experimentally determined values by proton NMR.

FIG. 3 shows dynamic light scattering analysis of example LNPs. FIG. 3A-FIG. 3C: Blank LNPs. Hydrodynamic radius (FIG. 3A), polydispersity (FIG. 3B), and particle size distribution (FIG. 3C) of various LNPs without mRNA. FIG. 3D-FIG. 3F: LNPs with mRNA. Hydrodynamic radius (FIG. 3D), polydispersity (FIG. 3E), and particle size distribution (FIG. 3F) of various LNPs with mRNA.

FIG. 4 shows characterization and quantification of encapsulated mRNA within example LNPs. FIG. 4A: gel electrophoresis of various LNPs before (−) and after (+) adding Triton X-100. FIG. 4B: schematic of Ribogreen assay. FIG. 4C: % mRNA encapsulation efficiency as measured by Ribogreen assay. *P<0.01, **P<0.001; Two-way ANOVA (Tukey's multiple comparison test).

FIG. 5 shows analysis of lipid ratios to improve luciferase mRNA encapsulation efficiency (EE) for example LNPs. FIG. 5A: EE of LNPs at various mol % of POEGMAL₁₀₋₅₀ and SM-102. Relation between EE and hydrodynamic radius of various LNPs at 0.5 (FIG. 5B), 1.5 (FIG. 5C), and 2.5 (FIG. 5D) mol %. FIG. 5E: EE of example LNPs after dialysis against PBS.

FIG. 6 shows analysis of luciferase mRNA EE of example LNPs at various mol % of POEGMAL; after dialysis against PBS. FIG. 6A: radius; FIG. 6B % polydispersity; and FIG. 6C % encapsulation.

FIG. 7 show cryogenic transmission electron microscopy (Cryo-TEM) images of LNP_{POEGMAL5} (top panel) and LNP_{POEGMAL10} (bottom panel) after dialysis against PBS

FIG. 8 shows analysis of parameters to improve EE of LNP_{POEGMAL10} to encapsulate therapeutically relevant mature full-length SAR COV-2 mRNA. EE before (FIG. 8A) and after (FIG. 8B) dialysis against PBS. FIG. 8C: Hydrodynamic radius after dialysis against indicated buffers. Effect of N:P (FIG. 8D), ethanol fraction during LNP preparation (FIG. 8E), and lipid mol % (FIG. 8F) on mRNA encapsulation.

FIG. 9 shows expression of Cluc mRNA cargo of example LNPs in HEK293T cells. FIG. 9A: relative expression with respect to Lipofectamine 2000 at 500 ng mRNA; FIG. 9B: relative expression with respect to Lipofectamine 2000 at 300 ng mRNA; FIG. 9C: raw AUC values at various N:P at 500 ng; and FIG. 9D: raw AUC values at various N:P at 300 ng.

FIG. 10 shows toxicity of LNPs against HEK293T cells after 48 h of continuous treatment.

FIG. 11 shows the expression of various amounts of CLuc mRNA cargo of example LNPs in HEK 293T cells at a charge ratio (N:P) ranging from 4:1 to 8:1, circle: LNP_{POEGMAL5}, square: LNP_{POEGMAL10}, and triangle: LN_{PEG-DMG}. FIG. 11A: 500 ng of mRNA at 8:1; FIG. 11B: 500 ng of mRNA at 6:1; FIG. 11C: 500 ng of mRNA at 4:1; FIG. 11D: 300 ng of mRNA at 8:1; FIG. 11E: 300 ng of mRNA at 6:1; and FIG. 11F: 300 ng of mRNA at 4:1.

FIG. 12 shows RNase protection assay. Example LNPs were incubated for 0.5 h after addition of RNase. The sequence of addition is indicated in the table on the left and the gel is on the right. All the example LNPs protect the Cluc mRNA cargo against RNase.

### DETAILED DESCRIPTION

Disclosed herein are POEGMAylated lipids that can be used to fabricate stealth LNPs for encapsulating therapeutics, such as full length model luciferase mRNA and therapeutically relevant SARS COV-2 mRNA, with more than 85% EE. Investigations of parameters of LNP production such as ethanol fraction, charge ratio (N:P), lipid mol %, and buffer exchange revealed how each parameter can affect EE of the LNPs. The POEGMAlyted LNPs also provided the necessary protection to mRNA against RNase that is a prerequisite to successful in vivo delivery. Further, in a reporter mRNA expression assay, LNPs with 10 kDa POEGMAlyted lipid outperformed a Moderna biosimilar LNP formulation. This is promising as it makes a strong case for POEGMAlyted LNP platform for in vivo mRNA vaccine delivery while potentially avoiding the immunogenicity of PEG.

### 1. DEFINITIONS

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed invention. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “antigen” refers to a molecule capable of being bound by an antibody or a T cell receptor. The term “antigen” also encompasses T-cell epitopes. An antigen is additionally capable of being recognized by the immune system and/or being capable of inducing a humoral immune response and/or cellular immune response leading to the activation of B-lymphocytes and/or T-lymphocytes. In some embodiments, the antigen contains or is linked to a Th cell epitope. An antigen can have one or more epitopes (B-epitopes and T-epitopes). Antigens may include polypeptides, polynucleotides, carbohydrates, lipids, small molecules, polymers, polymer conjugates, and combinations thereof. Antigens may also be mixtures of several individual antigens.

The term “antigenicity” refers to the ability of an antigen to specifically bind to a T cell receptor or antibody and includes the reactivity of an antigen toward pre-existing antibodies in a subject.

The term “effective amount” or “therapeutically effective amount” refers to an amount sufficient to effect beneficial or desirable biological and/or clinical results.

The term “immunogenicity” refers to the ability of an antigen to induce an immune response and includes the intrinsic ability of an antigen to generate antibodies in a subject. As used herein, the terms “antigenicity” and “immunogenicity” refer to different aspects of the immune system and are not interchangeable.

The term “mRNA,” as used herein, refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

The term “N:P ratio,” as used herein, refers to the molar ratio of ionizable (in the physiological pH range) nitrogen atoms in a lipid to phosphate groups in a nucleic acid (e.g., RNA)

As used herein, the term “nucleic acid” is used in its broadest sense and encompasses any compound and/or substance that includes a polymer of nucleotides. These polymers are often referred to as polynucleotides. Example nucleic acids or polynucleotides include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a b-D-ribo configuration, a-LNA having an a-L-ribo configuration (a diastereomer of LNA), 2′-amino-LNA having a 2′-amino functionalization, and 2′-amino-a-LNA having a 2′-amino functionalization) or hybrids thereof. Nucleic acids can be obtained by chemical synthesis methods or by recombinant methods.

The term “phospholipid,” as used herein, refers to a lipid that includes a phosphate moiety and one or more carbon chains, such as unsaturated fatty acid chains. A phospholipid may include one or more multiple (e.g., double or triple) bonds (e.g., one or more unsaturations).

The terms “polypeptide”, “peptide”, and “protein,” as used herein, may be used interchangeably to refer to a string of at least three amino acids linked together by peptide bonds. Peptides can contain natural amino acids, non-natural amino acids (i.e., compounds that do not occur in nature but that can be incorporated into a polypeptide chain), and/or amino acid analogs. Also, one or more of the amino acids in a peptide may be modified, for example, by the addition of a chemical entity such as a carbohydrate group, a phosphate group, a famesyl group, an isofarnesyl group, a fatty acid group, a linker for conjugation, functionalization, or other modification, etc. Modifications may include cyclization of the peptide, the incorporation of D-amino acids, etc.

The term “RNA,” as used herein, refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). RNAs may be selected from the non-limiting group consisting of small interfering RNA (siRNA), asymmetrical interfering RNA (aiRNA), microRNA (miRNA), Dicer-substrate RNA (dsRNA), small hairpin RNA (snRNA), mRNA, single-guide RNA (sgRNA), cas9 mRNA, and mixtures thereof.

The term “treatment” or “treating” refers to protection of a subject from a disease, such as preventing, suppressing, repressing, ameliorating, or completely eliminating the disease. Preventing the disease involves administering a conjugate of the present disclosure to a subject prior to onset of the disease. Suppressing the disease involves administering a conjugate of the present disclosure to a subject after induction of the disease but before its clinical appearance. Repressing or ameliorating the disease involves administering a conjugate of the present disclosure to a subject after clinical appearance of the disease.

The term “subject” includes humans and mammals (e.g., mice, rats, pigs, cats, dogs, and horses). Typical subjects of the present disclosure may include mammals, particularly primates and humans. For veterinary applications, suitable subjects may include, for example, livestock such as cattle, sheep, goats, cows, swine, and the like; poultry such as chickens, ducks, geese, turkeys, and the like, as well as domesticated animals particularly pets such as dogs and cats. For research applications, suitable subjects may include mammals, such as rodents (e.g., mice, rats, hamsters), rabbits, primates, and swine such as inbred pigs and the like.

### 2. LIPID NANOPARTICLES

Disclosed herein are lipid nanoparticles (LNPs) that include a POEGMA-lipid conjugate. The lipid nanoparticle can include an ionizable lipid, a phospholipid, a sterol, a POEGMA-lipid conjugate, and a therapeutic. The lipid nanoparticle can facilitate introduction of the therapeutic, such as a nucleic acid, into a cell, a tissue, an organ, a subject, or the like. The lipid nanoparticle can also include a targeting ligand that can facilitate interaction with a target cell. For example, the targeting ligand can specifically interact with a target cell (e.g., specifically interact with an extracellular protein on the surface of a target cell) to improve localization of the lipid nanoparticle following administration. Example targeting ligands include, but are not limited to, aptamers, carbohydrates, proteins, antibodies, single chain variable fragments, and the like In some embodiments, the lipid nanoparticle further includes a targeting ligand. In addition, the components of the lipid nanoparticle may be a pharmaceutically acceptable salt thereof.

When the therapeutic includes a nucleic acid, the amount of the lipids (e.g., ionizable lipid) and the amount of the nucleic acid can be selected to provide a specific N: P ratio. The N: P ratio of the lipid nanoparticle refers to the molar ratio of nitrogen atoms in one or more lipids to the number of phosphate groups in a nucleic acid. The one or more nucleic acids, lipids, and amounts thereof may be selected to provide an N: P ratio from about 2:1 to about 20:1, such as about 3:1 to about 19:1, about 4:1 to about 18:1, about 5:1 to about 17:1, about 4:1 to about 16:1, about 6:1 to about 16.1, about 7:1 to about 15:1, about 8:1 to about 14:1, about 9:1 to about 13:1, about 7:1 to about 12:1, about 5:1 to about 14:1, about 8:1 to about 12:1, about 2:1 to about 12:1, or about 3.1 to about 12:1. In some embodiments, the lipid nanoparticle has an N: P ratio of greater than 2:1, greater than 3:1, greater than 4:1, greater than 5:1, greater than 6:1, greater than 7:1, greater than 8:1, greater than 9:1, greater than 10:1, or greater than 11:1. In some embodiments, the lipid nanoparticle has an N: P ratio of less than 20:1, less than 19:1, less than 18:1, less than 17:1, less than 16:1, less than 15:1, less than 14:1, less than 13:1, less than 12:1, or less than 11:1. In some embodiments, the lipid nanoparticle has an N: P ratio of about 10:1. In some embodiments, the lipid nanoparticle has an N: P ratio of about 8:1.

The lipid nanoparticle can have a varying particle size that can depend on the lipid components that are included in the lipid nanoparticle. For example, the lipid nanoparticle can have a diameter of about 30 nm to about 300 nm, such as about 35 nm to about 250 nm, about 40 nm to about 200 nm, about 30 nm to about 150 nm, about 30 nm to about 100 nm, about 35 nm to about 90 nm, about 40 nm to about 80 nm, or about 35 nm to about 125 nm. In some embodiments, the lipid nanoparticle has a diameter of greater than 30 nm, greater than 35 nm, greater than 40 nm, greater than 45 nm, or greater than 50 nm. In some embodiments, the lipid nanoparticle has a diameter of less than 300 nm, less than 250 nm, less than 200 nm, less than 150 nm, or less than 100 nm.

The zeta potential of a lipid nanoparticle may be used to indicate the electrokinetic potential of the composition. For example, the zeta potential may describe the surface charge of a lipid nanoparticle. Lipid nanoparticles with relatively low charges, positive or negative, are generally desirable, as more highly charged species may interact undesirably with cells, tissues, and other elements in the body. The lipid nanoparticle can have a zeta potential of about −10 mV to about +20 mV, such as about −10 mV to about +15 mV, about −10 mV to about +10 mV, about −10 mV to about +5 mV, about 0 mV to about +5 mV, or about −5 mV to about +10 mV. In some embodiments, the lipid nanoparticle has a zeta potential of greater than −5 mV, greater than 0 mV, greater than +1 mV, greater than +2 mV, greater than +3 mV, or greater than +4 mV. In some embodiments, the lipid nanoparticle has a zeta potential of less than +20 mV, less than +19 mV, less than +18 mV, less than +17 mV, less than +16 mV, or less than +15 mV.

The efficiency of encapsulation of a therapeutic describes the amount of therapeutic that is encapsulated or otherwise associated with a lipid nanoparticle after preparation, relative to the initial amount provided. The encapsulation efficiency is desirably high. The encapsulation efficiency may be measured, for example, by comparing the amount of therapeutic in a solution containing the lipid nanoparticle before and after breaking up the lipid nanoparticle with one or more organic solvents or detergents. Fluorescence may be used to measure the amount of free therapeutic (e.g., RNA) in a solution. For the lipid nanoparticles described herein, the encapsulation efficiency of a therapeutic molecule may be greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, or greater than or equal to 99%, or about 100%. In some embodiments, the encapsulation efficiency is greater than or equal to 75%. In some embodiments, the encapsulation efficiency is greater than or equal to 85%. In some embodiments, the encapsulation efficiency is about 70% to about 99%, such as about 70% to about 90%, about 75% to about 95%, or about 75% to about 99%.

Lipid nanoparticles may be characterized by a variety of methods. For example, microscopy (e.g., transmission electron microscopy or scanning electron microscopy) may be used to examine the morphology and size distribution of a lipid nanoparticle. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a lipid nanoparticle, such as particle size, polydispersity index, and zeta potential.

Due to the inclusion of the POEGMA-lipid conjugate, compared to a PEG-lipid conjugate, the disclosed lipid nanoparticle may have advantageous immune response properties. For example, the lipid nanoparticle can have a reduced immune response relative to a lipid nanoparticle including PEG. The reduced or eliminated immune response can include a reduced or eliminated antigenicity, a reduced or eliminated immunogenicity, or both of the lipid nanoparticle. The beneficial immune interactions of the lipid nanoparticle can also be seen in that the lipid nanoparticle may not be reactive with pre-existing anti-PEG antibodies in a subject. Accordingly, the disclosed lipid nanoparticles can have beneficial interactions with a subject's immune system. Analysis of the lipid nanoparticle's interaction with a subject's immune system can be assessed as described in PCT/US2022/023158 (published as WO 2022/212911), which is incorporated by reference herein in its entirety.

The lipid nanoparticles can be made by a number of different techniques. An example technique includes an alcohol injection method. For example, the ionizable lipid, phospholipid, sterol, and POEGMA-lipid conjugate can be added to an alcohol to form a first mixture. The alcohol can be ethanol. The first mixture can be injected into a second mixture to provide a lipid nanoparticle mixture. The second mixture can include the therapeutic and a buffer (e.g., citrate buffer). The lipid nanoparticle mixture can be dialyzed after being provided.

### A. POEGMA-Lipid Conjugates

The POEGMA-lipid conjugate includes a POEGMA and a lipid. The POEGMA-lipid conjugate can instill the conjugate with advantageous stealth and immune system properties. The lipid nanoparticle can include one type of POEGMA-lipid conjugate (e.g., an individual conjugate) or can include at least 2, at least 3, at least 4, or at least 5 different types of POEGMA-lipid conjugates. In some embodiments, the lipid nanoparticle includes 2 to 5 different types of POEGMA-lipid conjugates. As an example, the lipid nanoparticle can include at least two different POEGMA-lipid conjugates that differ by the molecular weight of the POEGMA, the hydrocarbon length of the lipid, or both. These variations (e.g., in molecular weight and hydrocarbon chain length, as well as others) are discussed more below. The POEGMA and the lipid can be included in a 1:1 stoichiometric molar ratio. For example, the conjugate can include 1 POEGMA molecule attached to 1 lipid molecule.

The POEGMA has a poly(methyl methacrylate) backbone and a plurality of side chains covalently attached to the backbone. The side chains are oligomers of ethylene glycol (EG). For example, each side chain can include 2 to 9 monomers of EG repeated in tandem, such as 2 to 8 monomers of EG repeated in tandem, 2 to 7 monomers of EG repeated in tandem, 2 to 6 monomers of EG repeated in tandem, 2 to 5 monomers of EG repeated in tandem, or 2 to 4 monomers of EG repeated in tandem. In some embodiments, each side chain includes 3 monomers of EG repeated in tandem.

Adjacent side chains may be the same within the same POEGMA molecule or they may be different. For example, one side chain may have 3 monomers of EG repeated in tandem, while another side chain (in the same POEGMA molecule) may have 4 monomers of EG repeated in tandem

Each side chain can have a first terminal end and a second terminal end. The first terminal end can be covalently attached to the backbone. The second terminal end can be free. The second terminal end may be modified. In some embodiments, each second terminal end independently includes an alkyl, ester, amine, amide, or carboxyl group. In some embodiments, each second terminal end includes an alkyl. In some embodiments, each second terminal end includes a C₁-C₄ alkyl. In some embodiments, each second terminal end includes a methyl group. In some embodiments, each second terminal end does not include a hydroxyl group. The second terminal end of each side chain may be the same or different from the second terminal end of an adjacent side chain in the same POEGMA molecule. In some embodiments, the second terminal end of each side chain is the same throughout the POEGMA. In some embodiments, the second terminal end of at least one side chain is different from the second terminal end of at least one adjacent side chain.

In addition, the backbone can have a first terminal end and a second terminal end.

The POEGMA can have a varying molecular weight. For example, the POEGMA can have a number average molecular weight of about 1 kDa to about 100 kDa, such as about 1 kDa to about 85 kDa, about 1 kDa to about 75 kDa, about 1 kDa to about 60 kDa, about 1 kDa to about 50 kDa, about 2 kDa to about 45 kDa, about 3 kDa to about 40 kDa, about 4 kDa to about 35 kDa, about 5 kDa to about 30 kDa, about 1 kDa to about 30 kDa, about 1 kDa to about 25 kDa, about 1 kDa to about 20 kDa, about 1 kDa to about 15 kDa, about 1 kDa to about 12 kDa, or about 1 kDa to about 10 kDa. In some embodiments, the POEGMA has a number average molecular weight of greater than 1 kDa, greater than 2 kDa, greater than 3 kDa, greater than 4 kDa, greater than 5 kDa, greater than 6 kDa, greater than 7 kDa, greater than 8 kDa, greater than 9 kDa, or greater than 10 kDa. In some embodiments, the POEGMA has a number average molecular weight of less than 100 kDa, less than 90 kDa, less than 80 kDa, less than 70 kDa, less than 60 kDa, less than 50 kDa, less than 40 kDa, less than 30 kDa, less than 20 kDa, less than 15 kDa, less than 12 kDa, or less than 10 kDa. In some embodiments, the POEGMA has a number average molecular weight of about 10 kDa. Molecular weight of the POEGMA can be measured by techniques used within the art, such as SEC, SEC combined with multi-angle light scattering, gel permeation chromatography, and the like.

The lipid of the conjugate can be any suitable lipid that can be conjugated to the POEGMA and allow the conjugate thereof to be included in the lipid nanoparticle. The lipid can be saturated or unsaturated. The lipid can include varying lengths of a hydrocarbon chain. The number of carbon atoms in a hydrocarbon chain can be indicated by the prefix “C_{x-y}” or “Cₓ-C_{y}-”, wherein x is the minimum and y is the maximum number of carbon atoms in the hydrocarbon chain. Thus, for example, “C₆₋₂₂ hydrocarbon chain” or “C₆-C₂₂ hydrocarbon chain” refers to a hydrocarbon chain containing from 6 to 22 carbon atoms. The lipid may include a C₂₋₄₀ hydrocarbon chain, such as a C₂₋₃₅ hydrocarbon chain, a C₂₋₃₀ hydrocarbon chain, a C₂₋₂₅ hydrocarbon chain, a C₂₋₂₀ hydrocarbon chain, a C₄₋₄₀ hydrocarbon chain, a C₁₀₋₄₀ hydrocarbon chain, a C₆₋₂₂ hydrocarbon chain, a C₁₀₋₂₈ hydrocarbon chain, a C₁₂₋₂₀ hydrocarbon chain, a C₁₀₋₂₂ hydrocarbon chain, a C₁₂₋₃₀ hydrocarbon chain, a C₁₄₋₄₀ hydrocarbon chain, a C₁₂₋₁₈ hydrocarbon chain, or a C₈₋₁₈ hydrocarbon chain. In some embodiments, the lipid of the POEGMA-lipid conjugate has a hydrocarbon chain that is greater than 4 carbons in length, greater than 6 carbons in length, greater than 8 carbons in length, greater than 10 carbons in length, greater than 12 carbons in length, or greater than 14 carbons in length. In some embodiments, the lipid of the POEGMA-lipid conjugate has a hydrocarbon chain that is less than 40 carbons in length, less than 36 carbons in length, less than 32 carbons in length, less than 30 carbons in length, less than 24 carbons in length, or less than 20 carbons in length.

The lipid can include one hydrocarbon chain or a plurality of hydrocarbon chains. For example, the lipid can include 1 to 5 individual hydrocarbon chains, such as 1 to 4 individual hydrocarbon chains, 2 to 5 individual hydrocarbon chains, 1 to 3 individual hydrocarbon chains, or 2 to 4 individual hydrocarbon chains. In some embodiments, the lipid includes greater than 1 individual hydrocarbon chain, greater than 2 individual hydrocarbon chains, or greater than 3 individual hydrocarbon chains. In some embodiments, the lipid includes less than 5 individual hydrocarbon chains, less than 4 individual hydrocarbon chains, or less than 3 individual hydrocarbon chains. Lipids that have more than one hydrocarbon chain can have varying lengths of hydrocarbon chain as described above. In addition, embodiments that include a plurality of hydrocarbon chains can include individual hydrocarbon chains of all the same length or the lipid can include individual hydrocarbon chains of varying length.

The lipid can also include a number of different functional groups that can allow for flexibility in conjugating the lipid to the POEGMA. For example, the lipid can include a triazole, an amide, an ester, an ether, a hydrocarbon linker, and other suitable conjugation linkers. In some embodiments, the lipid is conjugated to the POEGMA through a triazole, an amide, an ester, an ether, or a hydrocarbon linker. In some embodiments, the lipid of the POEGMA-lipid conjugate includes 1,2-dimyristoyl-sn-glycerol, 1,2-distearoyl-sn-glycero-3-phosphoethanolamine, dipalmitoyl phosphatidylethanolamine, 1,2-dimyristyloxlpropyl-3-amine, or a combination thereof. Further discussion of different linker strategies are discussed below.

The POEGMA-lipid conjugate can be included in the lipid nanoparticle in varying amounts. For example, the lipid nanoparticle can include the POEGMA-lipid conjugate at about 0.1 mol % to about 10 mol %, such as about 0.2 mol % to about 9.5 mol %, about 0.3 mol % to about 9 mol %, about 0.4 mol % to about 8.5 mol %, about 0.1 mol % to about 8 mol %, about 0.1 mol % to about 7.5 mol %, about 0.1 mol % to about 7 mol %, about 0.1 mol % to about 6.5 mol %, about 0.1 mol % to about 6 mol %, about 0.1 mol % to about 5.5 mol %, about 0.1 mol % to about 5 mol %, about 0.2 mol % to about 7 mol %, about 0.2 mol % to about 6 mol %, about 0.3 mol % to about 6 mol %, about 0.3 mol % to about 5.5 mol %, about 0.1 mol % to about 5 mol %, about 0.1 mol % to about 4.5 mol %, about 0.1 mol % to about 4 mol %, about 0.1 mol % to about 3.5 mol %, about 0.1 mol % to about 3 mol %, about 0.1 mol % to about 2.5 mol %, about 0.1 mol % to about 2 mol %, about 0.1 mol % to about 1.5 mol %, about 0.1 mol % to about 1 mol %, or about 0.1 mol % to about 0.75 mol %. Mol % here and throughout refers to the molar percentage of a component, e.g., POEGMA-lipid conjugate here, as it relates to the total amount of lipid components of the lipid nanoparticle (e.g., ionizable lipid, phospholipid, sterol, and POEGMA-lipid conjugate).

In some embodiments, the lipid nanoparticle includes the POEGMA-lipid conjugate at greater than 0.1 mol %, greater than 0.15 mol %, greater than 0.2 mol %, greater than 0.25 mol %, greater than 0.3 mol %, greater than 0.35 mol %, greater than 0.4 mol %, greater than 0.45 mol %, greater than 0.5 mol %, greater than 1 mol %, greater than 2 mol %, greater than 3 mol %, greater than 4 mol %, or greater than 5 mol %. In some embodiments, the lipid nanoparticle includes the POEGMA-lipid conjugate at less than 10 mol %, less than 9.5 mol %, less than 9 mol %, less than 8.5 mol %, less than 8 mol %, less than 7.5 mol %, less than 7 mol %, less than 6.5 mol %, less than 6 mol %, less than 5.5 mol %, less than 5 mol %, less than 4.5 mol %, less than 4 mol %, less than 3.5 mol %, less than 3 mol %, less than 2.5 mol %, less than 2 mol %, less than 1.5 mol %, less than 1 mol %, less than 0.9 mol %, less than 0.8 mol %, less than 0.75 mol %, or less than 0.6 mol %.

The POEGMA can be conjugated to the lipid through any suitable conjugation strategy known within the art. For example, the lipid and the POEGMA may each individually have functional groups that are complimentary to each other in that they can form a covalent bond between the functional groups under appropriate conditions. Representative complimentary functional groups that can form a covalent bond include, but are not limited to, an amine and an activated ester, an amine and an isocyanate, an amine and an isothiocyanate, an amine and a carbonate, thiols for formation of disulfides, an aldehyde and amine for enamine formation, and an azide for formation of an amide via a Staudinger ligation. Functional groups suitable for conjugation also include bioorthogonal functional groups. Bioorthogonal functional groups can selectively react with a complementary bioorthogonal functional group. Bioorthogonal functional groups include, but are not limited to, an azide and alkyne for formation of a triazole via Click-chemistry reactions, trans-cyclooctene (TCO) and tetrazine (Tz) (e.g., 1,2,4,5-tetrazine), and others. In some embodiments, the lipid and the POEGMA each individually include bioorthogonal functional groups. In some embodiments, the lipid is functionalized with dibenzocyclooctyne, the POEGMA is functionalized with an azide, or both. Depending on the functional groups, different bonds or linkages can be formed between the lipid and the POEGMA. The POEGMA can be functionalized at its backbone or at a side chain.

Further discussion on POEGMA, its synthesis, and its application can be found in U.S. Pat. Nos. 8,497,356 and 10,364,451, both of which are incorporated herein by reference in their entirety.

### B. Ionizable Lipids

The lipid nanoparticle can include one or more ionizable lipids. “Ionizable lipid” (or alternatively cationic lipid) refers to a lipid having a positive or partial positive charge at physiological pH (e.g. pH of about 7.4). Ionizable lipids may also be zwitterionic, i.e., neutral molecules having both a positive and a negative charge The lipid nanoparticle can include one type of ionizable lipid (e g., an individual ionizable lipid) or can include at least 2, at least 3, at least 4, or at least 5 different types of ionizable lipids. In some embodiments, the lipid nanoparticle includes 2 to 5 different types of ionizable lipids.

Example ionizable lipids include, but are not limited to, (heptadecan-9-yl 8-((2-hydroxyethyl)(6-oxO)-6-(undecyloxy) hexyl) amino) octanoate) (SM-102), 3,6-bis({4-[bis(2-hydroxydodecyl)amino]butyl}) piperazine-2,5-dione (cKK-E12), 1-Linoleoyl-2-linoleyloxy-3-dmiethylaminopropane (DLin-2-DMAP), 1,2-Dilinoleylcarbanioyloxy-3-dimethylaniinopropane (DLin-C-DAP), 1,2-Dilmoleoyl-3-dimethylammopropane (DLm-DAP), 1,2-Dilinoleyloxy-N,N-dimethylaminopropane (DLin-DMA), 2,2-Dilinoleyl-4-dimethy laminomethy 1-[1,3]-dioxolane (DLin-K-DMA), 2,2-dilmoleyl-4-(2-dimethylaiiimoethyl)-[1,3]-dioxolane (DLin-KC2-DMA), (6Z,9Z,28Z,31Z)-heptatriaeonta-6,9,28,31-tetraen-19-yl 4-(dimethylamino)butanoate (DLin-MC3-DMA), 1,2-dioieoyl-3-dimethylammonium propane (DODAP), N,N-dimethyl-(2,3-dioleyloxy)propylamine (DODMA), dioctadecylamidoglycyocarboxyspermine (DOGS), spermine cholesterylcarbamate (GL-67), bis-guanidinium-spermidine-cholesterol (BGTC), 3b-(N-(N^{A}N′-dimethylammoethanej-carbam oxl cholesterol (DC-Chol), N-t-butyl-N′-tetradecylamino-propionamidine (diC14-amidine), dimethyldioctadecylammoniumbromide (DDAB), N-(1,2-dimyristyloxyprop-3-yl)-N,N-dimethyl-N-hydroxyethyl ammonium bromide (DMR1E), N,N-dioleyl-N,N-dimethylammonium chloride (DODAC), dioleyloxypropyl-3-dimethyl hydroxy ethyl ammonium bromide (DORIE), N-(1-(2,3-dioleyloxy3)propyl)-N-2-(spenninecarboxamido)ethyl)-N,N-dimethylamrnonium trifluoracetate (DOSPA), 2-dioleoy trimethyl ammonium propane chloride (DOTAP), N-(1-(2 3-dioleyloxy)propyl)-N N,N-trimethylammonium chloride (DOTMA), aminopropyl-dimethyl-bis(dodecyloxy)-propanaminiumbromide (GAP-DLRIE), 1,2-dioleoyl-sn-3-phosphoethanolamine (DOPE), and (4-Hydroxybutyl)azanediyl]di(hexane-6,1-diyl)bis(2-hexyldecanoate) (ALC-0315).

In some embodiments, the ionizable lipid includes (heptadecan-9-yl 8-((2-hydroxyethyl)(6-oxO-6-(undecyloxy) hexyl) amino) octanoate) (SM-102), (4-Hydroxybutyl)azanediyl]di(hexane-6,1-diyl)bis(2-hexyldecanoate) (ALC-0315), or a combination thereof. In some embodiments, the ionizable lipid includes (heptadecan-9-yl 8-((2-hydroxyethyl) (6-oxO-6-(undecyloxy) hexyl) amino) octanoate) (SM-102).

The ionizable lipid can be included in the lipid nanoparticle in varying amounts. For example, the lipid nanoparticle can include the ionizable lipid at about 20 mol % to about 65 mol %, such as about 25 mol % to about 60 mol %, about 25 mol % to about 65 mol %, about 30 mol % to about 65 mol %, about 40 mol % to about 65 mol %, about 45 mol % to about 65 mol %, about 20 mol % to about 55 mol %, about 20 mol % to about 50 mol %, about 20 mol % to about 45 mol %, about 30 mol % to about 60 mol %, about 35 mol % to about 55 mol %, about 20 mol % to about 65 mol %, about 40 mol % to about 55 mol %, or about 45 mol % to about 55 mol %. In some embodiments, the lipid nanoparticle includes the ionizable lipid at greater than 20 mol %, greater than 25 mol %, greater than 30 mol %, greater than 35 mol %, greater than 40 mol %, greater than 45 mol %, greater than 50 mol %, or greater than 55 mol %. In some embodiments, the lipid nanoparticle includes the ionizable lipid at less than 65 mol %, less than 60 mol %, less than 58 mol %, less than 56 mol %, less than 54 mol %, less than 52 mol %, less than 50 mol %, or less than 45 mol %.

### C. Phospholipids

The lipid nanoparticle can include one or more phospholipids. Generally, phospholipids may include a phospholipid moiety and one or more fatty acid moieties. The lipid nanoparticle can include one type of phospholipid (e.g., an individual phospholipid) or can include at least 2, at least 3, at least 4, or at least 5 different types of phospholipids. In some embodiments, the lipid nanoparticle includes 2 to 5 different types of phospholipids.

Example phospholipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanol amine (D SPE), monomethyl-phosphatidylethanolamine, dimethyl-phosphatidylethanolamine, 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilinolcoyl-sn-glycero-3-phosphocholine (DLPC), 1,2-dimyristoyl-sn-glycero-3-phosphocholine (DMPC), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, and dilinoleoylphosphatidylcholine.

In some embodiments, the phospholipoid includes DSPC, DPPE, DMPG, DOPC, DPPC, DOPG, or a combination thereof. In some embodiments, the phospholipid includes DSPC, DPPE, DOPC, or a combination thereof. In some embodiments, the phospholipid includes DSPC. In some embodiments, the phospholipid is DSPC.

The phospholipid can be included in the lipid nanoparticle at varying amounts. For example, the lipid nanoparticle can include the phospholipid at about 5 mol % to about 25 mol %, such as about 6 mol % to about 20 mol %, about 7 mol % to about 18 mol %, about 8 mol % to about 16 mol %, about 5 mol % to about 20 mol %, about 5 mol % to about 15 mol %, about 6 mol % to about 15 mol %, about 6 mol % to about 12 mol %, about 8 mol % to about 12 mol %, or about 9 mol % to about 11 mol %. In some embodiments, the lipid nanoparticle includes the phospholipid at greater than 5 mol %, greater than 6 mol %, greater than 7 mol %, greater than 8 mol %, greater than 9 mol %, greater than 10 mol %, or greater than 15 mol %. In some embodiments, the lipid nanoparticle includes the phospholipid at less than 20 mol %, less than 19 mol %, less than 18 mol %, less than 17 mol %, less than 16 mol %, less than 15 mol %, less than 14 mol %, less than 13 mol %, less than 12 mol %, less than 11 mol %, or less than 10 mol %.

### D. Sterols

The lipid nanoparticle can include one or more sterols. The term “sterol” refers to a subgroup of steroids also known as steroid alcohols. Sterols are usually divided into two classes: (1) plant sterols also known as “phytosterols”, and (2) animal sterols also known as “zoosterols.” The lipid nanoparticle can include one type of sterol (e.g., an individual sterol) or can include at least 2, at least 3, at least 4, or at least 5 different types of sterols. In some embodiments, the lipid nanoparticle includes 2 to 5 different types of sterols. In some embodiments, the sterol comprises a zoosterol.

Examples of sterols include, but are not limited to, cholesterol, campesterol, antrosterol, desmosterol, nicasterol, stigmasterol, sitosterol, oxysterol, C₄₋₁₀ sterol, ergosterol, and cholest-4-en-3-one. In some embodiments, the sterol includes cholesterol, campesterol, antrosterol, desmosterol, nicasterol, stigmasterol, sitosterol, oxysterol, C₄₋₁₀ sterol, ergosterol, cholest-4-en-3-one, or a combination thereof. In some embodiments, the sterol includes cholesterol, campesterol, stigmasterol, sitosterol, C₄₋₁₀ sterol, ergosterol, cholest-4-en-3-one, or a combination thereof.

In some embodiments, the sterol comprises cholesterol. In some embodiments, the sterol is cholesterol. The cholesterol can be cholesterol itself or a salt or ester thereof, e.g., cholesterol succinic acid, cholesterol sulfate, cholesterol hemisuccinate, cholesterol phthalate, cholesterol phosphate, cholesterol valerate, cholesterol acetate, cholesteryl oleate, cholesteryl linoleate, cholesteryl myristate, cholesteryl palmitate, cholesteryl arachidate, or cholesteryl phosphorylcholine.

The sterol can include a derivative of cholesterol. Example derivatives of cholesterol, include, but are not limited to, dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 3β[N-(N′N′-dimethylaminoethyl) carbamoyl cholesterol (DC-Chol), 24 (S)-hydroxycholesterol, 25-hydroxycholesterol, 25(R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, Sa-cholest-7-en-3β-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol, and fecosterol, or a salt or ester thereof.

The sterol can be included in the lipid nanoparticle at varying amounts. For example, the lipid nanoparticle can include the sterol at about 10 mol % to about 50 mol %, such as about 15 mol % to about 45 mol %, about 20 mol % to about 40 mol %, about 25 mol % to about 40 mol %, about 30 mol % to about 40 mol %, about 35 mol % to about 45 mol %, about 35 mol % to about 40 mol %, about 20 mol % to about 50 mol %, about 25 mol % to about 50 mol %, about 30 mol % to about 50 mol %, about 15 mol % to about 40 mol %, or about 15 mol % to about 35 mol %. In some embodiments, the lipid nanoparticle includes the sterol at greater than 10 mol %, greater than 15 mol %, greater than 20 mol %, greater than 25 mol %, greater than 30 mol %, or greater than 35 mol %. In some embodiments, the lipid nanoparticle includes the sterol at less than 50 mol %, less than 45 mol %, less than 42 mol %, less than 40 mol %, less than 38 mol %, or less than 35 mol %.

### E. Therapeutics

The lipid nanoparticle can include one or more therapeutics. Example therapeutics include, but are not limited to, nucleic acids and anionic polypeptides. In some embodiments, the lipid nanoparticle includes a nucleic acid, an anionic polypeptide, or both. In some embodiments, the lipid nanoparticle includes a nucleic acid or an anionic polypeptide.

The lipid nanoparticle can include one or more nucleic acids. A nucleic acid can be employed for the production of, e.g., of a polypeptide in a cell. Examples of nucleic acids include, but are not limited to, siRNA, miRNA, antisense oligonucleotides, shRNA, mRNA, ERNA, IRNA, CircRNA, and DNA. In some embodiments, the nucleic acid includes siRNA, miRNA, antisense oligonucleotides, shRNA, mRNA, tRNA, rRNA, CircRNA, DNA or a combination thereof. In some embodiments, the nucleic acid includes siRNA, miRNA, antisense oligonucleotides, shRNA, mRNA, tRNA, IRNA, CircRNA, or DNA. In some embodiments, the nucleic acid includes siRNA, miRNA, antisense oligonucleotides, shRNA, mRNA, tRNA, rRNA, or CircRNA. In some embodiments, the nucleic acid includes siRNA, mRNA, or a combination thereof. In some embodiments, the nucleic acid includes siRNA or mRNA. In some embodiments, the nucleic acid includes mRNA. In some embodiments, the nucleic acid is mRNA. An mRNA may encode a polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell.

The nucleic acid can be an RNA. Examples of RNA include, but are not limited to, messenger RNAs (mRNAs) (e.g., encoding a protein of interest), modified mRNAs (mmRNAs), mRNAs that incorporate a micro-RNA binding site(s) (miR binding site(s)), modified RNAs that comprise functional RNA elements, microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNA), locked nucleic acids (LNAs) and CRISPR/Cas9 technology.

In some embodiments, the nucleic acid includes SEQ ID NO: 1, SEQ ID NO: 2, or a combination thereof. In some embodiments, the nucleic acid includes SEQ ID NO: 1 or SEQ ID NO: 2.

Nucleic acids and anionic polypeptides can be commercially purchased. Alternatively, nucleic acids can be prepared by in vitro transcription from a DNA template. Techniques and methods of providing nucleic acids from a DNA template can be done via techniques known within the art and such as those described in the Examples. The nucleic acid can be modified before application by stabilizing sequences, capping, and polyadenylation. In addition, anionic polypeptides can be prepared via chemical synthesis and/or recombinant methods

In some embodiments, the lipid nanoparticle includes (heptadecan-9-yl 8-((2-hydroxyethyl)(6-oxO-6-(undecyloxy) hexyl) amino) octanoate) (SM-102); DSPC; cholesterol; a poly[oligo (ethylene glycol) ether methacrylate] (POEGMA)-lipid conjugate at about 0.25 mol % to about 3 mol %, wherein the POEGMA has a number average molecular weight of about 1 kDa to about 50 kDa; and mRNA.

### 3. PHARMACEUTICAL COMPOSITIONS

Further disclosed herein are pharmaceutical compositions that include one or more lipid nanoparticles. The pharmaceutical composition can further include a pharmaceutically acceptable excipient. The term “pharmaceutically acceptable excipient,” as used herein, means a non-toxic, inert solid, semi-solid or liquid filler, diluent, encapsulating material or formulation auxiliary of any type. Some examples of materials which can serve as pharmaceutically acceptable excipients are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols, such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, citrate buffers, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator. The route by which the composition is administered and the form of the composition can dictate the type of excipient to be used.

The pharmaceutically acceptable excipient may make up greater than 50% of the total mass or volume of a pharmaceutical composition including a lipid nanoparticle(s). For example, the pharmaceutically acceptable excipient may make up about 50%, about 60%, about 70%, about 80%, about 90%, or more of a pharmaceutical composition. In some embodiments, a pharmaceutically acceptable excipient is at least 95%, at least 96%, at least 97%, at least 98%, at least 99%, or 100% pure. In some embodiments, the pharmaceutically acceptable excipient is approved for use in humans and for veterinary use. In some embodiments, the pharmaceutically acceptable excipient is approved by United States Food and Drug Administration. In some embodiments, the pharmaceutically acceptable excipient is pharmaceutical grade. In some embodiments, the pharmaceutically acceptable excipient meets the standards of the United States Pharmacopoeia (USP), the European Pharmacopoeia (EP), the British Pharmacopoeia, and/or the International Pharmacopoeia.

General guidelines for the formulation and manufacture of pharmaceutical compositions and agents are available, for example, in Remington's The Science and Practice of Pharmacy, 21st Edition, A. R. Gennaro; Lippincott, Williams & Wilkins, Baltimore, Md., 2006, which is incorporated by reference herein in its entirety. Conventional excipients and accessory ingredients may be used in any pharmaceutical composition, except insofar as any conventional excipient or accessory ingredient may be incompatible with one or more components of a lipid nanoparticle. An excipient or accessory ingredient may be incompatible with a component of a lipid nanoparticle if its combination with the component may result in any undesirable biological effect or otherwise deleterious effect.

In some embodiments, the pharmaceutically acceptable excipient includes buffering agents, solubilizers, solvents, antimicrobial preservatives, antioxidants, suspension agents, a tablet or capsule diluent, a tablet disintegrant, or a combination thereof. In some embodiments, the pharmaceutically acceptable excipient includes buffering agents, solubilizers, solvents, antimicrobial preservatives, antioxidants, suspension agents, a tablet or capsule diluent, or a tablet disintegrant.

In some embodiments, the pharmaceutically acceptable excipient includes a buffer. In some embodiments, the buffer includes citrate and an alcohol. In some embodiments, the buffer is an about 70% to about 80% mM citrate-ethanol buffer. In some embodiments, the buffer is dialyzed against another buffer. In some embodiments, the buffer is dialyzed against a tris-acetate buffer.

The pharmaceutical compositions may be suitable for administration to a subject (such as a patient, which may be a human or non-human) well known to those skilled in the pharmaceutical art. The pharmaceutical composition may be prepared for administration to a subject. Such pharmaceutical compositions can be administered in dosages and by techniques well known to those skilled in the medical arts taking into consideration such factors as the age, sex, weight, and condition of the particular subject, and the route of administration.

The composition can be administered prophylactically or therapeutically. In prophylactic administration, the composition can be administered in an amount sufficient to induce a response. In therapeutic applications, the composition can be administered to a subject in need thereof in an amount sufficient to elicit a therapeutic effect. An amount adequate to accomplish this is defined as “therapeutically effective dose.” Amounts effective for this use will depend on, e.g., the particular composition of the conjugate regimen administered, the manner of administration, the stage and severity of the disease, the general state of health of the patient, and the judgment of the prescribing physician.

The composition may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, and subjects treated, the particular compounds employed, and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies.

Dosage amount and interval may be adjusted individually to provide plasma levels of the biologically active agent which are sufficient to maintain the modulating effects, or minimal effective concentration (MEC). The MEC will vary for each agent but can be estimated from in vivo and/or in vitro data. Dosages necessary to achieve the MEC will depend on individual characteristics and route of administration. However, assays well known to those in the art can be used to determine plasma concentrations. Dosage intervals can also be determined using MEC value. Compositions can be administered using a regimen which maintains plasma levels above the MEC for 10-90% of the time, such as between 30-90% or between 50-90%. In cases of local administration or selective uptake, the effective local concentration of the drug may not be related to plasma concentration.

The pharmaceutical composition can be administered at varying dosages depending on, e.g., different characteristics of the subject and the route of administration. In some embodiments, pharmaceutical compositions of the disclosure may be administered at dosage levels sufficient to deliver about 0.0001 mg/kg to about 10 mg/kg, about 0.001 mg/kg to about 10 mg/kg, about 0.005 mg/kg to about 10 mg/kg, about 0.01 mg/kg to about 10 mg/kg, about 0.1 mg/kg to about 10 mg/kg, about 1 mg/kg to about 10 mg/kg, about 2 mg/kg to about 10 mg/kg, about 5 mg/kg to about 10 mg/kg, about 0.0001 mg/kg to about 5 mg/kg, about 0.001 mg/kg to about 5 mg/kg, about 0.005 mg/kg to about 5 mg/kg, about 0.01 mg/kg to about 5 mg/kg, about 0.1 mg/kg to about 10 mg/kg, about 1 mg/kg to about 5 mg/kg, about 2 mg/kg to about 5 mg/kg, about 0.0001 mg/kg to about 1 mg/kg, about 0.001 mg/kg to about 1 mg/kg, about 0.005 mg/kg to about 1 mg/kg, about 0.01 mg/kg to about 1 mg/kg, or about 0.1 mg/kg to about 1 mg/kg in a given dose, where a dose of 1 mg/kg provides 1 mg of therapeutic or lipid nanoparticle per 1 kg of subject body weight. In some embodiments, a dose of about 0.005 mg/kg to about 5 mg/kg of therapeutic or lipid nanoparticle of the disclosure may be administrated.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine

### 4. METHODS

Also disclosed herein are methods of using the lipid nanoparticles and pharmaceutical compositions thereof. The description of the lipid nanoparticles, POEGMA-lipid conjugates, ionizable lipids, phospholipids, sterols, therapeutics, and pharmaceutical compositions can also be applied to the methods of treating and delivering a therapeutic to a cell disclosed herein.

### A. Methods of Treating a Disease or Disorder

Further provided are methods of treating a disease or disorder in a subject in need thereof. The method may include administering to the subject an effective amount of one or more lipid nanoparticles as disclosed herein. The lipid nanoparticle(s) may be administered optionally in combination with a pharmaceutically acceptable excipient (e.g., as a disclosed pharmaceutical composition).

The disclosed lipid nanoparticle can facilitate the production of polypeptides within a cell, a tissue, an organ, or a subject. Accordingly, a number of diseases or disorders can benefit from this ability. Example diseases or disorders include, but are not limited to, an infectious disease, Huntington's disease, muscular dystrophy, an autoimmune disease, and cancer. In some embodiments, the disease or disorder is an infection disease, cancer, or an autoimmune disease. In some embodiments, the disease or disorder is an infectious disease, such as a virus.

In some embodiments, the method can modulate an immune response with a subject suffering from an infectious disease, to thereby enhance an immune response against the infectious disease pathogen in the subject. Non-limiting examples of infectious diseases that can be treated include those caused by viral, bacterial, fungal, yeast and parasitic pathogens. Example viruses include, but are not limited to, Influenza Type A and B virus, Zika, Rabies, RSV, Chikungunya, cyclomegalovirus, Human Metapneumovirus, Ebola, HIV-1, and SARS-CoV-2.

The methods can be used to modulate an immune response, e.g., in a manner as done by a vaccine. In some embodiments, the nucleic acid of the lipid nanoparticle can provide a polypeptide that can stimulate the activation or activity of an immune cell, such as a dendritic cell or myeloid cell. For example, the nucleic acid can encode a polypeptide that is an antigen, such as a vaccine antigen (e.g., viral antigen, bacterial antigen, tumor antigen). In some embodiments, the nucleic acid (e.g., mRNA) associated with/encapsulated by the lipid nanoparticle encodes an antigen of interest, such as a cancer antigen or an infectious disease antigen (e.g., a bacterial antigen, a viral antigen, a fungal antigen, a protozoa antigen or a parasite antigen).

In some embodiments, the method is used for stimulating an immune response with a subject suffering from cancer to thereby enhance an immune response against the cancer in the subject. Non-limiting examples of cancers that can be treated include adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, myelodysplastic syndrome (including refractory anemias and refractory cytopenias), myeloproliferative neoplasms or diseases (including polycythemia vera, essential thrombocytosis and primary myelofibrosis), liver cancer (e.g., hepatocellular carcinoma), non-small cell lung cancer, small cell lung cancer, lung carcinoid tumor, lymphoma of the skin, malignant mesothelioma, multiple myeloma, myelodysplasia syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor, and secondary cancers caused by cancer treatment.

In some embodiments, the method is used to modulate an immune response with a subject having aberrant immune activity, including subjects suffering from an autoimmune disease, an allergic disorder or an inflammatory response. Nonlimiting examples of autoimmune diseases that can be treated include rheumatoid arthritis, systemic lupus erythematosus, inflammatory bowel disease (including ulcerative colitis and Crohn's disease), Type 1 diabetes, multiple sclerosis, psoriasis, Graves' disease, Hashimoto's thyroiditis, chronic inflammatory demyelinating polyneuropathy, Guillain-Barre syndrome, myasthenia gravis, glomerulonephritis and vasculitis

As discussed elsewhere, the lipid nanoparticles and pharmaceutical compositions thereof can have advantageous immune properties. For example, following administration, the lipid nanoparticle and pharmaceutical composition thereof can have a reduced immune response relative to a lipid nanoparticle including PEG; may not react with pre-existing anti-PEG antibodies in the subject; or a combination thereof.

### B. Methods of Delivering a Therapeutic to a Cell

Also provided are methods of delivering a therapeutic to a cell. The method may include delivering a therapeutic to a cell by contacting the cell with one or more lipid nanoparticles or a pharmaceutical composition thereof as disclosed herein. In some embodiments, the therapeutic is a nucleic acid. By contacting the cell with the lipid nanoparticle(s), the particle can be internalized by e.g., endocytosis, and the nucleic acid (e.g., mRNA) may be translated in the cell to produce a polypeptide of interest.

Contacting the cell may be done in vivo, ex vivo, in culture, or in vitro. The amount of lipid nanoparticle contacted with a cell, and/or the amount of therapeutic therein, may depend on the type of cell or tissue being contacted, the means of administration, the physiochemical characteristics of the lipid nanoparticle and the therapeutic (e.g., size, charge, and chemical composition) therein, and other factors. An effective amount of the lipid nanoparticle or pharmaceutical composition thereof can allow for efficient polypeptide production in the cell. Metrics for efficiency may include polypeptide translation (indicated by polypeptide expression), level of mRNA degradation, and/or immune response indicators.

The type of cell that can be targeted or delivered to is generally not limiting. Accordingly, a myriad of cell types can be used in the methods disclosed herein. Example cells include, but are not limited to, hepatocytes, epithelial cells, hematopoietic cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells.

As described elsewhere the nucleic acid (e.g., mRNA) included in the lipid nanoparticle can encode a polypeptide that is an antigen, such as a vaccine antigen (e.g, viral antigen, bacterial antigen, tumor antigen). In some embodiments, the nucleic acid associated with/encapsulated by the lipid nanoparticle encodes an antigen, such as a cancer antigen or an infectious disease antigen.

In some embodiments, the nucleic acid included in the lipid nanoparticle may encode a recombinant polypeptide that may replace one or more polypeptides that may be substantially absent in a cell contacted with the lipid nanoparticle(s). The one or more substantially absent polypeptides may be lacking due to a genetic mutation of the encoding gene or a regulatory pathway thereof. Alternatively, a recombinant polypeptide produced by translation of the nucleic acid may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In some embodiments, a recombinant polypeptide produced by translation of the nucleic acid may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the nucleic acid may be engineered for localization within the cell, such as within a specific compartment such as the nucleus or may be engineered for secretion from the cell or for translocation to the plasma membrane of the cell.

As the methods of treating a disease or disorder may include delivering a therapeutic to a cell, the description of methods of delivering a therapeutic to a cell may also be applied to methods of treating a disease or disorder. Likewise, the description of methods of treating a disease or disorder can be applied (when applicable) to the methods of delivering a therapeutic to a cell.

The disclosed invention has multiple aspects, illustrated by the following non-limiting examples.

### 5. EXAMPLES

### Example 1

### Materials & Methods

General Materials. All the chemicals were purchased from Millipore Sigma (St. Louis, MO) unless otherwise specified. All the solvents were purchased from VWR International (Radnor, PA). 1,2-dipalmitoyl-sn-glycero-3-phosphoethanolamine-N-dibenzocyclooctyl (16 DBCO PE), 1,2-distearoyl-sn-glycero-3-phosphocholine (DSPC), and Cholesterol were procured from Avanti Polar Lipids (Birmingham, AL). (heptadecan-9-yl 8-((2-hydroxyethyl) (6-oxO-6-(undecyloxy) hexyl) amino) octanoate) [SM-102] was obtained from SINOPEG (China). pCMV-CLuc2 encoding Cypridina luciferase (CLuc) gene, HiScribe™ T7 ARCA mRNA Kit, and Monarch® RNA Cleanup Kit was purchased from New England BioLabs (Ipswich, MA). Quant-it™ RiboGreen RNA Assay kit, Pierce™ Cypridina Luciferase Glow Assay Kit, Lipofectamine 2000, and Citrate buffer was obtained from Thermo Fisher. LDH-Glo™ Cytotoxicity Assay kit and proteinase K were procured from Promega.

Synthesis of azido POEGMA (POEGMA₅₋₅₀). Triethylene glycol methyl ether methacrylate was passed through basic alumina to remove free radical inhibitors. Copper(II) bromide and tris(2-pyridylmethyl) amine (TPMA) were mixed in ultrapure water to prepare the catalytic complex with final concentration of 0.01 M CuBr₂ and 0.08 M TPMA. In a schlenk flask placed on an ice bath, the following were added: triethylene glycol methyl ether methacrylate (10 mmol, 2.26 mL), methanol (5.8 mL), Cu catalytic complex (0.01 mmol, 1 mL), water-NaCl solution (2 mmol, 4 ml from 0.5 M), azidoethyl-2-bromoisobutyrate (0.1 mmol, 16.4 μL) and the flask was sealed with a septum. In a separate schlenk flask, a fresh solution of ascorbic acid in ultrapure water (64 mM, 2 mL) was prepared. Both the flasks were kept cold in an ice bath and purged with argon for 45 minutes to remove oxygen. After deoxygenation, the ascorbic acid solution was injected continuously into the polymerization flask at a rate of 0.001 mL/min using a syringe pump under an inert atmosphere for different times to get varied molecular weight. The resulting solution was purged with air for an hour to quench the reaction and then dialyzed against water for 4 days. The dialyzed solution was then freeze dried to obtain azido POEGMA as a viscous gel which was stored at −20° C. Conditions to synthesize different MW azido POEGMAs are given below in Table 1:

TABLE 1
Reaction conditions of different MW azido POEGMAs

| | Molar ratio | | Reaction |
|---|---|---|---|
| Polymers | monomer | initiator | time (min) |
| POEGMA₅ | 50 | 1 | 75 |
| POEGMA₁₀ | 100 | 1 | 90 |
| POEGMA₂₀ | 150 | 1 | 140 |
| POEGMA₄₀ | 200 | 1 | 180 |
| POEGMA₅₀ | 200 | 1 | 210 |

Gel permeation chromatography-multi-angle light scattering (GPC-MALS). GPC-MALS was used to characterize the size and dispersity of the POEGMA polymer. All the GPC experiments were performed at room temperature on two in-line columns (Agilent PLgel mixed-C column 105 Å, 7.5×300 mm, 5 μm, part number PL1110-6500) using THF (stabilized with 100 ppm BHT) as eluent. The flow rate of 1 ml/min was set using an Agilent 1260 Infinity Isocratic pump. Molecular weights were determined using an in-line Wyatt-DAWN TREOS MALS detector and Wyatt-Optilab DSP refractive index detector. The refractive index increment (dn/dc) values were determined by using an online 100% mass recovery assumption method built into Wyatt Astra software for known concentrations and masses of the samples. UV-absorbance was measured with an in-line Agilent 1260 Infinity UV detector. All the samples for GPC-MALS analysis were prepared by dissolving 2 mg of a sample into 1 ml of HPLC grade THF and filtered through 0.2 μm pore size Inorganic membrane syringe filter (Whatman, Anotop™ 10).

Synthesis of POEGMAylated Lipids (POEGMAL₅₋₅₀). 100 mg of azido POEGMA₁₀₋₅₀ was dissolved in chloroform at a 20 mg/mL concentration. A 5-molar excess of 16 DBCO PE was added and the solution was incubated in a 37° C. shaker for 24 h. The reaction was monitored with thin layer chromatography using 10% methanol in chloroform (v/v) as mobile phase. After 24 h the reaction mixture was evaporated and reprecipitated from 10% chloroform hexane mixture except for POEGMAL₅. For POEGMALs 10% methanol hexane mixture was used. 3-4 rounds of reprecipitation resulted in pure POEGMAylated Lipids with >90% yield.

Reverse Phase High Performance Liquid Chromatography (HPLC). Purity of POEGMA₅₋₅₀ was assessed by reverse phase HPLC using Phenomenex C18 column as stationary phase and methanol at 1 mL/min as mobile phase. Compound was detected using an in-line UV-detector at 230 nm.

NMR Spectroscopy. NMR was performed on a Bruker 16.4 Tesla spectrometer (Bruker, UK) with a BBO room temperature probe. POEGMAL₅₋₅₀ was solubilized in CDCl₃ and the solutions were investigated by 1D-dimensional ¹H spectroscopy analyses. Data was processed using MestReNova x64.

Synthesis of mRNA and Agarose gel Electrophoresis. In vitro transcribed mRNA, encoding Cypridina luciferase (CLuc) gene was synthesized using HiScribe™ T7 ARCA mRNA Kit (with tailing) (NEB cat no: E2060S) following manufacturer's protocol. Briefly, plasmid pCMV-CLuc2 (NEB cat no: N0321), encoding the reporter gene, luciferase, was linearized by XbaI (20 units/μg DNA) for 30 min at 37° C. and purified using Oligo Clean & Concentrator spin columns (Zymo Research cat no. D4060). Linearization of plasmid downstream of the gene avoids generation of long heterogenous transcripts by T7 RNA polymerase. A 20 μL in vitro transcription (IVT) reaction was set with 1 μg linearized plasmid, 2 μL T7 RNA polymerase mix and 1X ribonucleotide mix (1 mM GTP, 4 mM anti-reverse cap analog, 1.25 mM CTP, 1.25 mM UTP, >1.25 mM ATP final). The reaction was incubated at 37° C. for 30 min. DNA template in IVT reaction was then digested using DNaseI at the concentration of 0.2 U/μL and incubated at 37° C. for 30 min. Poly(A) tailing was then performed in 1X poly (A) buffer using 5 μL Poly (A) polymerase in 100 μL reaction. The reaction was incubated at 37° C. for 30 min. Synthesized mature mRNA was purified using the Monarch® RNA Cleanup Kit (NEB cat no: T2050) and eluted in 20 μL RNA storage buffer (Invitrogen cat no: AM7001).

DNA template for SARS-COV2 spike protein mRNA was assembled in-house using Gibson assembly. The gblocks encoding Wuhan strain SARS-COV-2 spike protein (1-1273, K986P and V987P) along with untranslated regions (UTRs) were assembled into pCMV vector along with UTRs. Specifically, to improve mRNA stability and translation efficiency, (i) human alpha-globin 5′ UTR with Kozak sequence was incorporated upstream of the protein coding sequence, (ii) 3′ UTRs derived from mitochondrially encoded 12S rRNA (mtRNR1) and amino-terminal enhancer of split (AES) was incorporated downstream in tandem and (iii) for co-transcriptional tailing, a poly-A tail interrupted by a 10 nt linker (A30LA70, L=GCAUAUGACU) was incorporated at 3′end of the template. These elements were selected based on the mRNA sequence accessed from WHO International “Non-proprietary Names Programme”. The T7 promoter sequence was also modified for co-transcriptional capping with CleanCap-AG (Cap1) and a BbsI site was introduced for scarless run-off transcription. The template sequence was verified by Sanger Sequencing and plasmid was transformed into NEB-5-alpha competent cells. pCMV-SARS-Cov2s plasmid was purified using Qiagen's plasmid purification kit and linearized using BbsI. A typical IVT reaction included T7 RNA polymerase, inorganic pyrophosphatase, ribonuclease inhibitor at concentrations recommended by the manufacturer (Aldevron), nucleotide triphosphates (NTPs, 2.5 mM or 5 mM each), Cleancap AG (3′ OMe) (80% of GTP) and MgCl₂ in 1X transcription buffer (40 mM HEPES-KOH (pH=7.5), 2 mM spermidine, 10 mM DTT) and was incubated at 37° C. for 2 h or 4 h. To minimize byproducts, Mg:NTP ratio, total NTP concentration and incubation time was optimized. mRNA was purified using NEB's Monarch mRNA purification kit and quantified using Nanodrop. mRNA quality was verified by 1% agarose gel electrophoresis run at 130 mV for 30 min in TAE buffer after adding the RNA loading dye and denaturing the samples at 65° C. for 10 min. The gel was imaged by SyBr safe staining.

Formulation of Lipid Nanoparticles (LNPs). LNPs were prepared by using widely used ethanol injection method (see Duong et al., Preparation of Solid Lipid Nanoparticles and Nanostructured Lipid Carriers for Drug Delivery and the Effects of Preparation Parameters of Solvent Injection Method. Molecules 2020, 25 (20), 1-36 and Ganesan et al., Lipid Nanoparticles: Different Preparation Techniques, Characterization, Hurdles, and Strategies for the Production of Solid Lipid Nanoparticles and Nanostructured Lipid Carriers for Oral Drug Delivery. Sustain. Chem. Pharm. 2017, 6, 37-56, both of which are incorporated by reference herein in their entirety). Briefly, Ionisable lipid SM-102, DSPC, cholesterol, and stealth lipid (PEG-DMG or POEGMAL₅₋₅₀) were dissolved in ethanol at 50:10:38.5:0.5-2.5 mol %. The ethanolic solution containing the above lipid mixture (one volume) was rapidly injected into four volumes of 10 mM citrate buffer (pH 4) containing mRNA at 4:1-8:1 nitrogen (from SM-102) to phosphorous (from mRNA) molar ratio (N:P). Unless otherwise mentioned LNP_{POEGMAL5-50} were prepared at N:P 8:1 while LNP_{PEG-DMG} was prepared at 6:1. The resulting solution was dialyzed against PBS for 24 h and stored at 4° C.

Buffer Compositions. The following buffers were used for dialysis of the mRNA loaded LNP_{POEGMAL(n)} and LNP_{PEG-DMG}: A) Pfizer: 10 mM Tris buffer, 300 mM sucrose, pH of 7.4. B) Moderna: Tris (0.5 mg/mL), Tris-HCl (2.5 mg/mL), glacial acetic acid (0.042 mg/mL), sodium acetate trihydrate (0.2 mg/mL), sucrose (87 mg/mL).

Dynamic Light Scattering (DLS) Study The hydrodynamic radius (Rₕ) and polydispersity of all the LNPs were determined by dynamic light scattering (DLS) using a temperature programmed DynaPro microsampler (Wyatt Technology, Santa Barbara, CA). Samples were prepared in PBS and filtered (0.2 μm cellulose filters) into a black 96-well plate with flat clear bottom. At least 15 acquisitions were taken at 25° C., and the collected data were analyzed by a regularization fit of the autocorrelation function using DYNAMICS v7 software (Wyatt technology).

Ribogreen assay. Ribogreen assay was performed on non-dialyzed samples. LNPs were assessed for mRNA with or without 1% Triton X-100 in Tris-EDTA buffer using Ribogreen assay kit according to manufacturer's protocol. LNP samples were incubated with 1% Triton X-100 for 5 min at RT to allow disruption of LNPs. Encapsulation efficiency was calculated by using the formulae: %Encapsulation=(RFUf−RFUi)/RFUf*100 where RFUi and RFUf is relative fluorescence unit before and after adding Tritonx-100, respectively, to the LNPs

Cell Culture HEK293T cells were obtained from Duke University Core Culture facility. Cells were cultured in Dulbecco's Modified Eagle's Medium (DMEM) supplemented with 10% Fetal Bovine Serum (heat inactivated, Gibco) and 2 mM L-glutamine (Gibco). Cells were maintained in 5% CO₂ incubator at 37° C.

Luciferase expression assay. The assay was performed using Pierce™ Cypridina Luciferase Glow Assay Kit (Thermo Scientific) as per the manufacturer's recommended protocol. Briefly, HEK293T (1×10⁴ cells/well) were plated in 96-well plate and incubated overnight at 37° C. in 5% CO₂. The media was removed, and cells were treated with 300 ng or 500 ng mRNA prepared in 100 μL Opti-MEM media/well and incubated at 37° C. in 5% CO₂. Lipofectamine 2000 was used as per the manufacturer's protocol. Expression of luciferase was monitored at predetermined time points by adding 5 uL of media from transfected cells to 25 uL of 1X Vargulin in the assay buffer provided with the kit. After 10 min incubation at room temperature, luminescence was quantified at 463 nm using BioTek's Synergy H1 hybrid multi-mode reader.

Cytotoxicity assay. HEK293T cells were seeded in 96 well plate at 3000 cells/well 16 h prior to treatment. Cells were treated with various LNPs in a range of concentration. 48 h after treatment released lactate dehydrogenase (LDH) from the cells with compromised membrane was assessed with LDH-glo assay kit as per the manufacturer instruction.

RNase protection assay. LNP containing 300 ng of CLuc mRNA was incubated with 333 pg RNase/ug of mRNA, before or after addition of 0.1% final concentration of Triton-X, for 2 h at 37° C. Finally, the RNase was quenched by incubating the samples with 1X proteinase K in 50 mM TrisHCl/CaCl₂ buffer for 20 min at 55° C. Samples were run on a 1% agarose gel as described earlier.

### Example 2

### Synthesis and Characterization of Azido POEGMA (POEGMA₅₋₅₀)

POEGMAylated lipid (POEGMAL) was synthesized using click conjugation of two building blocks: the POEGMA polymer and a fatty acid tail. The DBCO-modified fatty acid tail (16:0 DBCO PE) was commercially obtained while the azide modified POEGMA polymer was synthesized in-house (Scheme 1). Briefly, copper(II)-tris(2-pyridylmethyl) amine (TPMA) catalytic complex was incubated with triethylene glycol methyl ether methacrylate and azidoethyl-2-bromoisobutyrate in an ice bath under inert atmosphere. Reaction was initiated by addition of a fresh solution of ascorbic acid continuously into the flask. Upon completion, the reaction was quenched by purging the mixture with air. Azido POEGMA was purified by dialyzing against water and stored as a lyophilized viscous gel.

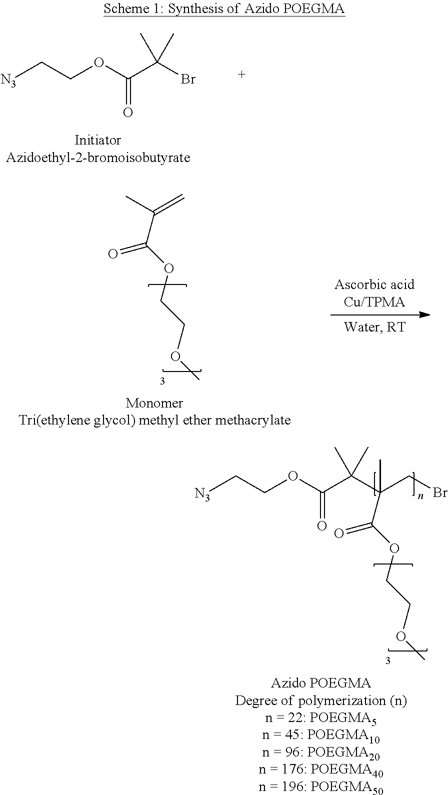

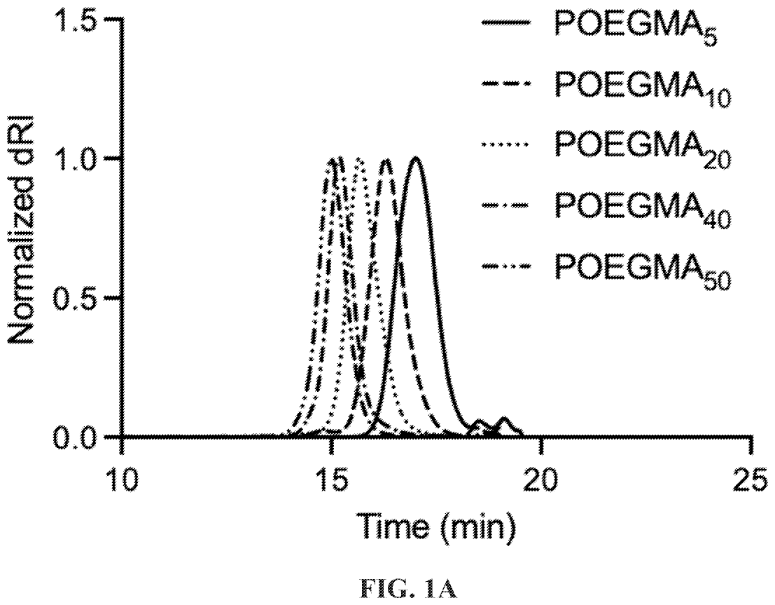

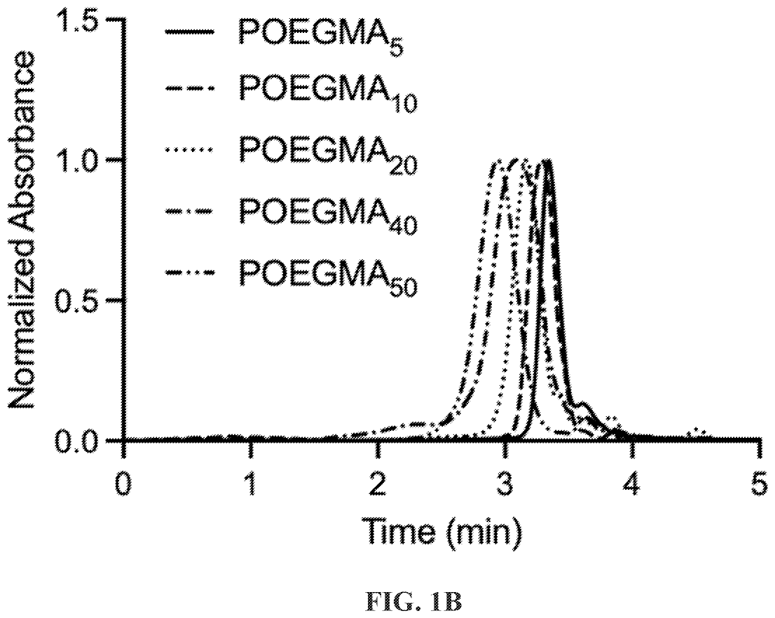

Five variants of azido POEGMA were synthesized with MW ranging from 5-50 kDa. Number-averaged molecular weight (Mₙ), weight-averaged molecular weight (M_{w}), and polydispersity (Ð) of azido POEGMA were assessed by gel permeation chromatography-multi-angle light scattering (GPC-MALS). Results from the GPC-MALS confirmed the size and narrow polydispersity of azido POEGMA (Table 2 & FIG. 1A). The degrees of polymerization (n) were calculated by subtracting M_{w} of the polymerization initiator from M_{w} of POEGMA and dividing the resulting mass by the average M_{w} of the monomeric unit (Table 2). The purity of the azido POEGMA was also assessed with reverse phase high performance liquid chromatography (HPLC). The HPLC trace of all the POEGMA at 230 nm confirmed greater than 95% purity (FIG. 1B). The structure was further confirmed with ¹H NMR by calculating the integral ratios of various protons, which closely aligned with the expected structures. Finally, azide incorporation was confirmed by reacting azido-POEGMA with excess DBCO-PEG₄. DBCO has a characteristic absorbance at 308 nm which is proportional to its concentration. As DBCO reacts with the azido group of the POEGMA polymer with near 100% efficiency, calculation of concentration of the unreacted DBCO enables the determination of the concentration for the azide in the reaction mixture. Reaction of azido POEGMA₅₋₅₀ with DBCO-PEG₄ at 44 h indicated greater than 90% azide content in all POEGMA polymers.

TABLE 2
Characterization of POEGMA by GPC-MALS

| Azido | | | | |
|---|---|---|---|---|
| POEGMA | Mₙ (kDa) | M_{w} (kDa) | Ð | n |
| POEGMA₅ | 4.8 | 5.3 | 1.10 | 22 |
| POEGMA₁₀ | 9.1 | 10.6 | 1.16 | 45 |
| POEGMA₂₀ | 20.4 | 22.5 | 1.11 | 96 |
| POEGMA₄₀ | 36.9 | 41.0 | 1.11 | 176 |
| POEGMA₅₀ | 41.6 | 45.7 | 1.10 | 196 |

### Example 3

### Synthesis and Characterization of POEGMA-Lipids (POEGMAL₅₋₅₀)

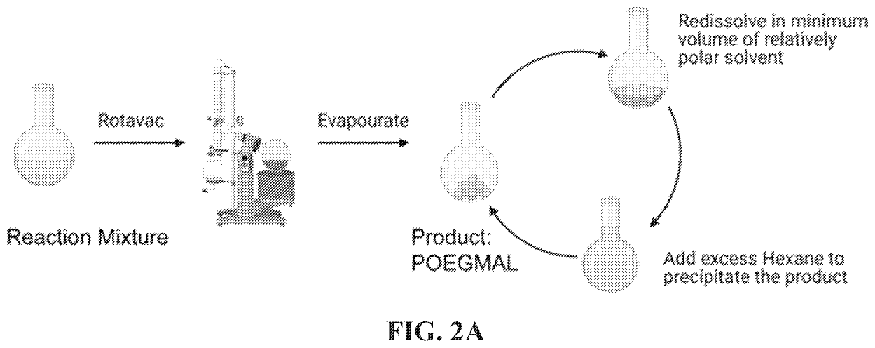

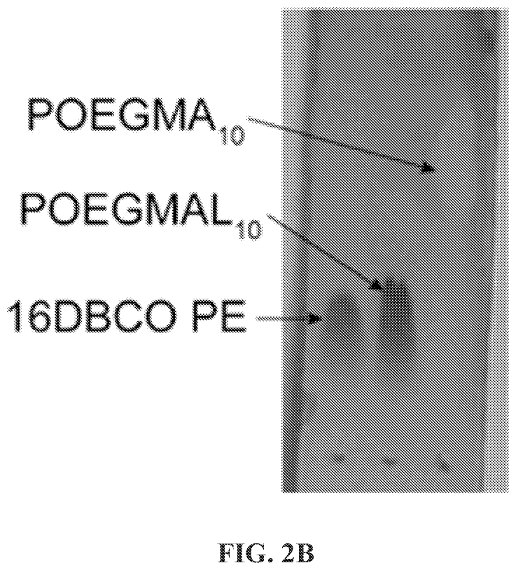

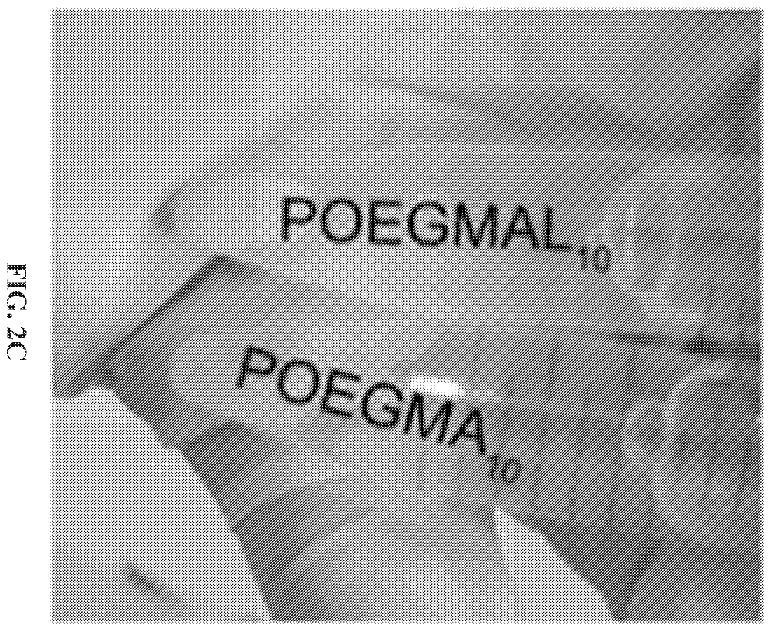

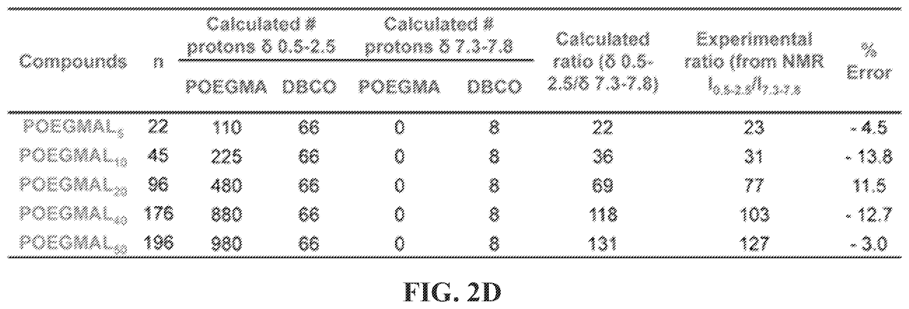

The azido POEGMA₅₋₅₀ was click conjugated with 5-molar excess of 16DBCO PE in chloroform by strain promoted azide-alkyne cycloaddition (SPAAC) to obtain POEGMA-Lipid conjugates (POEGMAL₅₋₅₀). Twenty-four hours after the reaction, the product was purified by reprecipitating from methanol for 5 kDa or chloroform for 10-50 kDa POEGMAL with excess hexane (FIG. 2A). The pellet was finally redissolved in methanol and subjected to reverse phase HPLC. A typical thin layer chromatographic plate is shown in FIG. 2B. POEGMAL₁₀ migrated more slowly than POEGMA₁₀. Also, the typical physical appearance of the azido POEGMA is transparent while that of POEGMAL is opaque (FIG. 2C). The absence of unreacted POEGMA₅₋₅₀ peak in the HPLC trace indicates POEGMA₅₋₅₀ and 16 DBCO PE were completely consumed in the respective reaction. As expected, the retention time of the POEGMAL increases compared to POEGMA after addition of hydrophobic lipid residue.

To further confirm the conjugation, ¹H NMR analysis was performed. The NMR spectrogram of final POEGMA-Lipid conjugates possess the trace of both POEGMA₅₋₅₀ and 16DBCO PE indicating successful conjugation. The presence of aromatic protons at δ˜7.3-7.8 ppm in the POEGMAL₅₋₅₀ suggests successful integration of DBCO modified fatty acid tail to the POEGMA backbone. POEGMA₅₋₅₀ and 16DBCO PE lipid has several overlapping regions which are difficult to deconvolute and only the aromatic region (Chemical shift δ˜7.3-7.8 ppm) of 16DBCO PE is far apart from this overlapping region. Hence, the integration ratio between δ˜0.5-2.5 ppm and between 7.3-7.8 (I_{0.5-2.5}/I_{7.3-7.8}) was used to calculate proton ratios (FIG. 2D). The difference between the theoretical and experimental ratios is presumably because (i) the numbers of protons are greater in δ˜0.5-2.5 ppm than δ˜7.3-7.8 ppm which can suppress the sensitivity; and/or (ii) the residual solvent peak in δ˜7.3-7.8 ppm region may interfere with the calculation. Nevertheless, collectively all these data strongly suggest successful synthesis and purification of POEGMAylated lipids.

### Example 4

### Formulation and Characterization of Lipid Nanoparticle (LNPs)

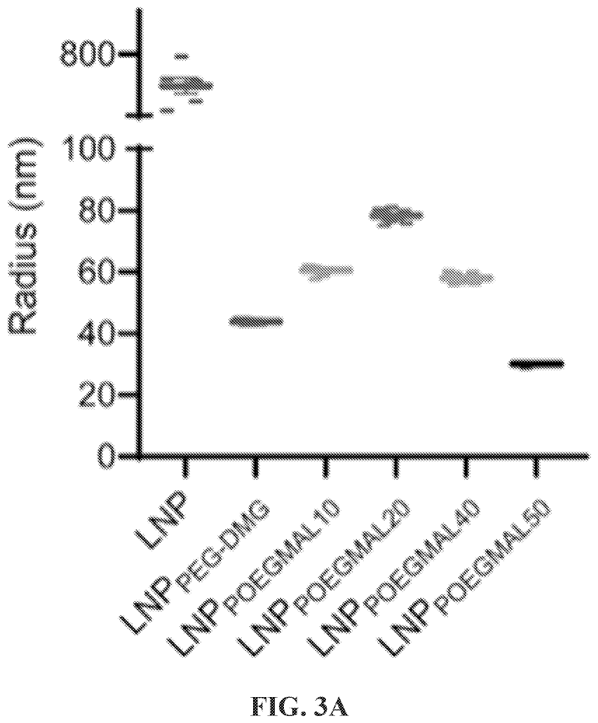

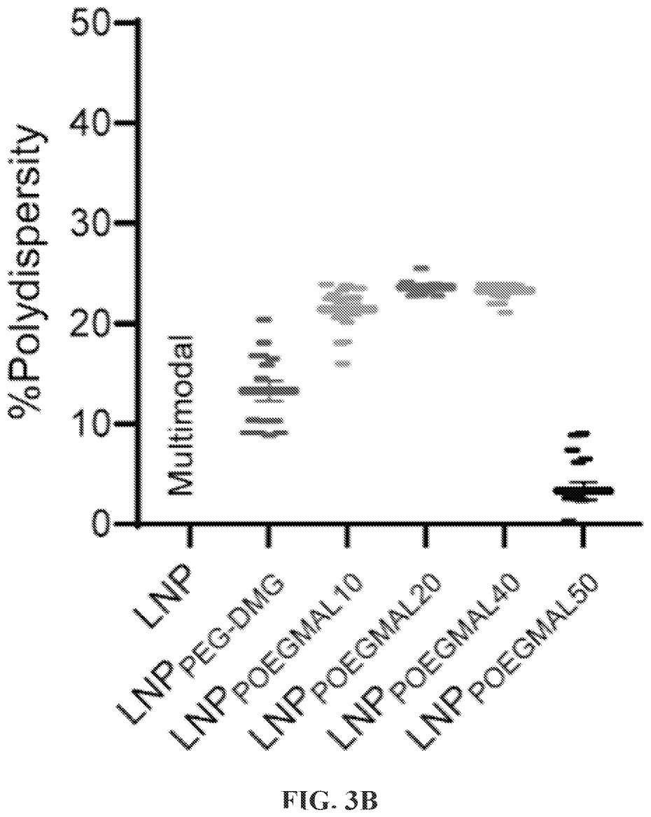

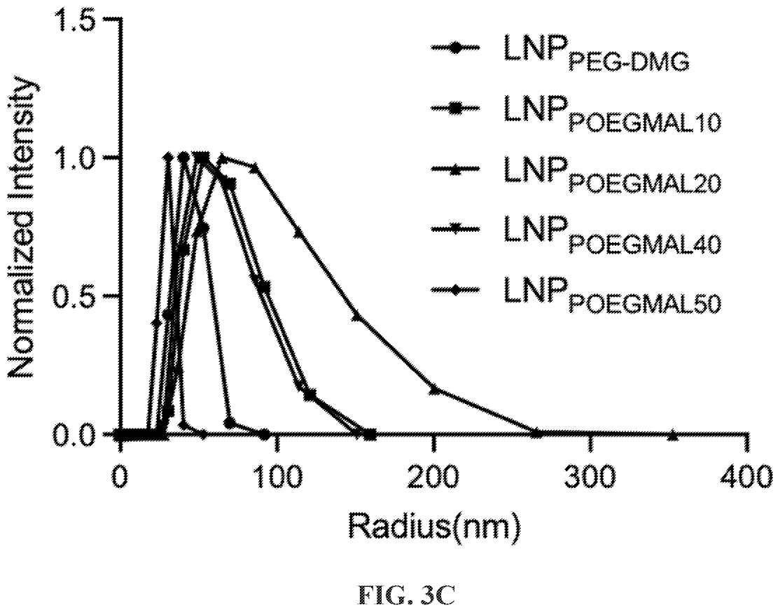

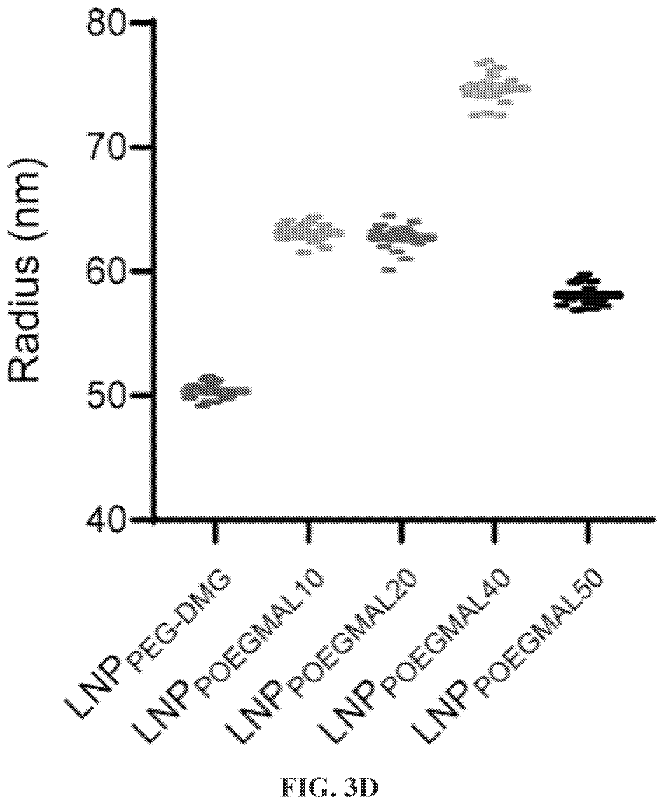

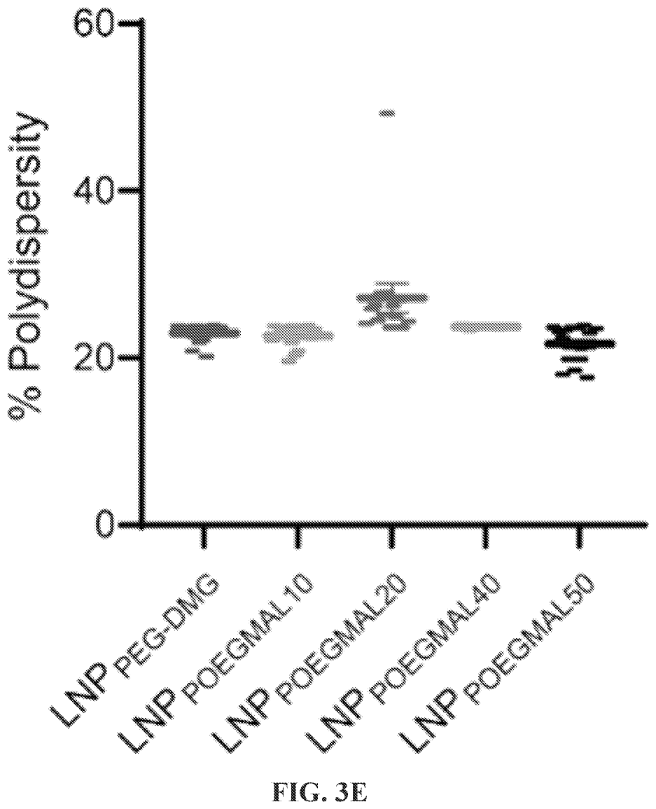

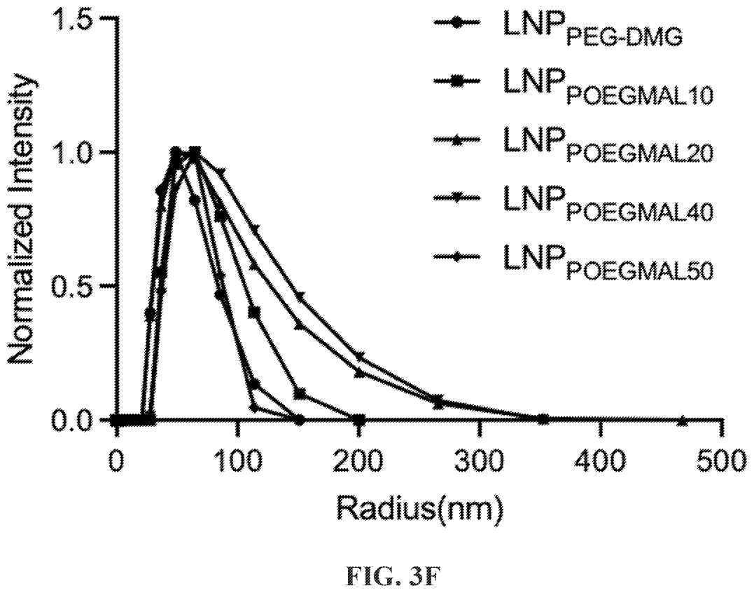

LNPs were formulated using ethanol injection methods with four different co-lipids with % mol indicated in parentheses: (i) an ionizable lipid (SM102) that helps to encapsulate mRNA, provides stability, and helps the particle for successful endosomal escape (50 mol %); (ii) DSPC (10 mol %); (iii) Cholesterol (38.5 mol %); and (iv) a stealth lipid (1.5 mol %) which is either a commercially available PEG-DMG or in-house synthesized POEGMAylated lipid This lipid ratio was specifically tailored for PEGylated lipids and provided a good starting point to formulate LNPs with POEGMAylated lipids. The LNP formulation with PEG-DMG acts as a benchmark and this lipid composition was also used in Moderna's mRNA COVID19 vaccine. It is noted that 10-50 kDa POEGMAL was used for the initial investigation of the role of MW and lipid ratios on mRNA encapsulation efficiency of the LNPs and incorporated POEGMAL5 in later part of the study. The lipids solubilized in small volume of ethanol was rapidly injected into citrate buffer (pH 4) containing luciferase mRNA to obtain the LNPs. The LNPs were then buffer exchanged with PBS at 4° C. A set of LNPs without any mRNA was also synthesised. Dynamic light scattering (DLS) was used to characterize the formation of nanoparticles. As summarized in FIG. 3A and FIG. 3B, stable LNPs were obtained when a stealth lipid was used. LNP without PEG-or POEGMA-lyted lipids appeared cloudier, had high polydispersity, and displayed an Rₕ>600 nm. In contrast, all other LNPs were translucent and had sub 100 nm Rₕ with narrow polydispersity (FIG. 3A, FIG. 3B, and FIG. 3C). Encapsulation of mRNA did not change the size of the nanoparticle significantly for PEG-DMG, POEGMAL₁₀, and POEGMAL₂₀. The Rₕ of LNPs with POEGMAL₄₀ and POEGMAL₅₀ the increased after mRNA encapsulation by 1.3-fold and 1.9-fold, respectively (FIG. 3D, FIG. 3E, and FIG. 3F).

### Example 5

Quantification of mRNA Encapsulation Efficiency

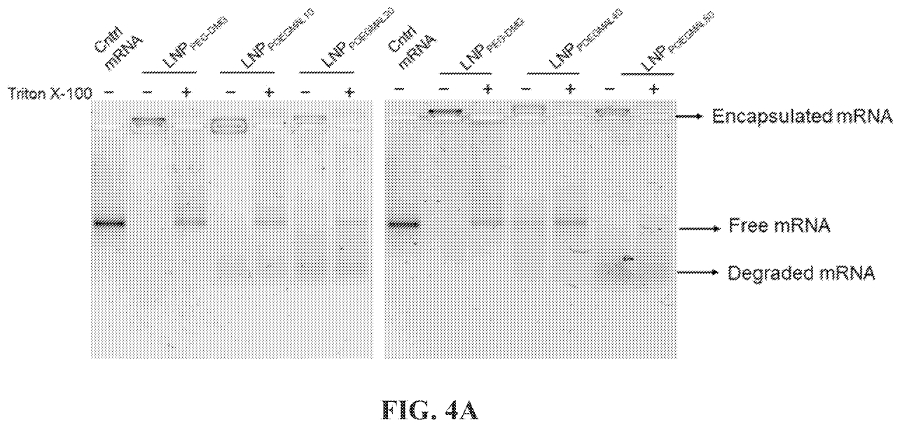

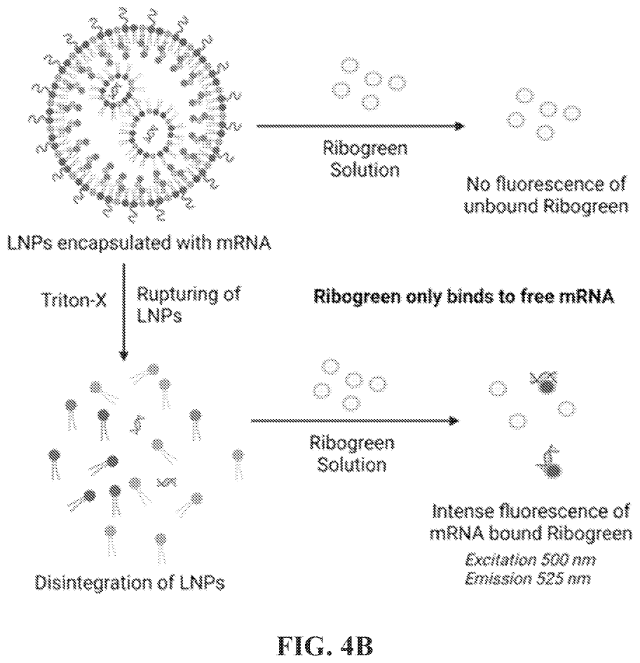

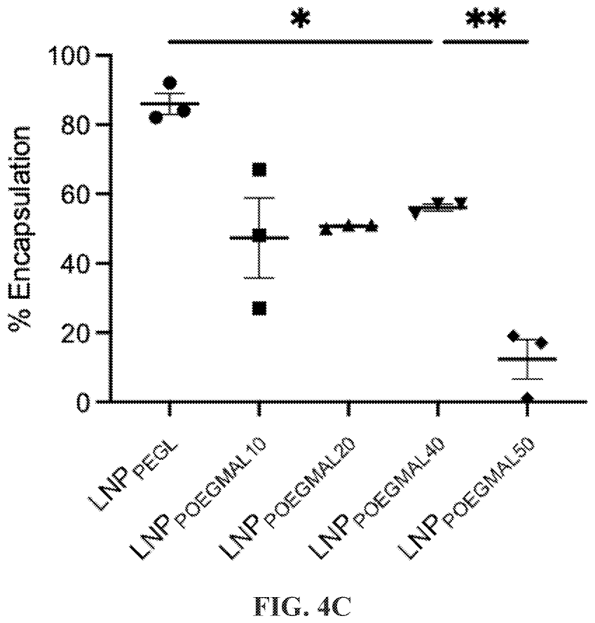

The mRNA encapsulation efficiency of the LNPs was first qualitatively assessed using agarose electrophoretic mobility shift assay (EMSA). As only free mRNA migrates through the agarose gel—but not the LNP mRNA—the free mRNA from the LNP mRNA in a formulation can be resolved. The total amount of mRNA—encapsulated and free—was estimated on an agarose gel by rupturing the LNPs with a surfactant such as Triton-X, which releases the encapsulated mRNA. The free mRNA at equivalent concentration acts as a control in the experiment. The electrophoresis experiment also indicates any degradation of mRNA during preparation and storage. As can be seen from FIG. 4A, all the LNPs encapsulated appreciable amount of mRNA. LNP_{POEGMAL40} and LNP_{PEG-DMG} Significantly reduced degradation of mRNA during handling and storage. On the other hand, mRNA was degraded in the case of LNP_{POEGMAL50}. For LNP_{POEGMAL10-20}, only free mRNA was degraded but not the encapsulated mRNA. This indicates the unencapsulated mRNA in case of LNP_{POEGMAL40} is not totally free in solution but rather it is associated weakly with the LNPs, otherwise it would have been degraded similarly as in the case of LNP_{POEGMAL10-20}. Next, the percent encapsulation of mRNA within the POEGMAylated LNPs was quantified with Ribogreen assay and benchmarked with that of PEGylated LNPs. To nullify the effect of handling and storage the encapsulation immediately after preparation without dialyzing the formulation was assessed. DLS confirmed that the size of the LNPs remains similar both before dialysis and after dialysis. Ribogreen is an organic dye that only binds to free mRNA but not the mRNA associated with the LNPs or the degraded nucleotides. In unbound state Ribogreen possesses little to no fluorescence but exhibits intense fluorescence when bound to free mRNA (FIG. 4B). The relative fluorescence unit (RFU) of Ribogreen mixed with LNPs is proportional to the amount of free mRNA in the LNP solution whereas the RFU of Ribogreen in LNPs ruptured with 1% Triton-X is proportional to total mRNA (sum of both free and encapsulated). The relative fluorescence absorbance of Ribogreen thus can be used to calculate % encapsulation. As evidenced from FIG. 4C, the encapsulation efficiency of LNP_{POEGMAL50} was lowest ˜12%. In contrast the encapsulation efficiency of LNPs increased by ˜4.5-fold for LNP_{POEGMAL40}, 4.1-fold for LNP_{POEGMAL20}, and 3.8-fold for LNP_{POEGMAL10} compared to LNP_{POEGMAL50}. The LNPs containing the POEGMAL₄₀ showed an appreciable encapsulation of 56% which is only 1.5-fold lower than the PEG-DMG. This lower encapsulation efficiency can be expected as the lipid ratios used to prepare the LNPs are tailored for PEGylated LNPs.

### Example 6

### Analysis and Characterization of LNPs to Maximize mRNA Encapsulation

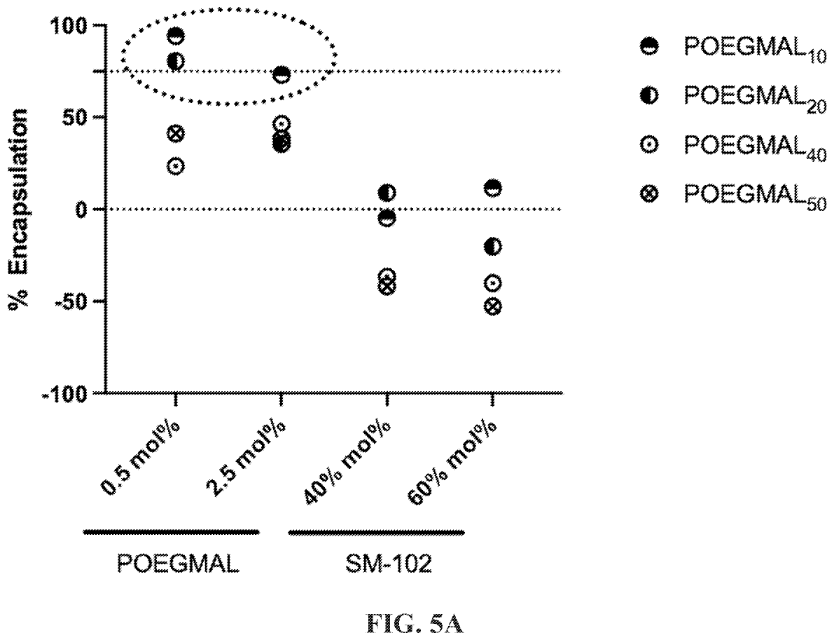

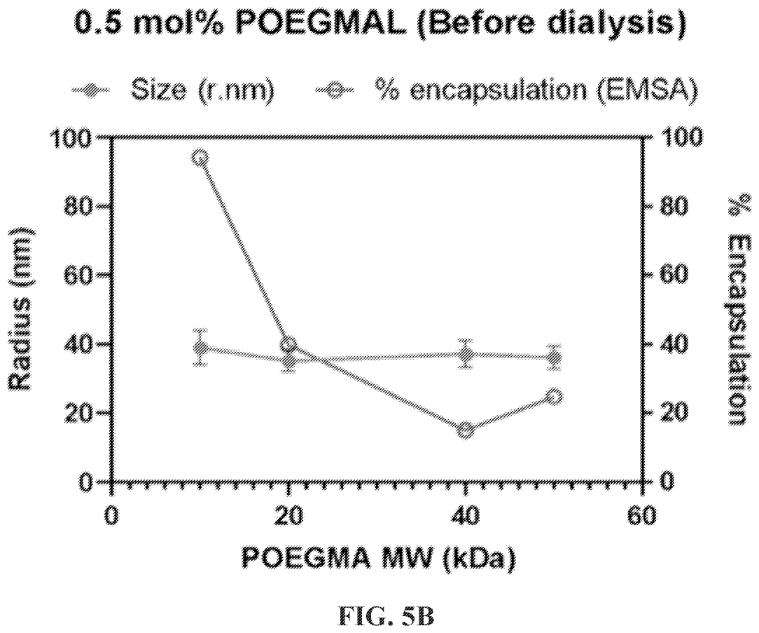

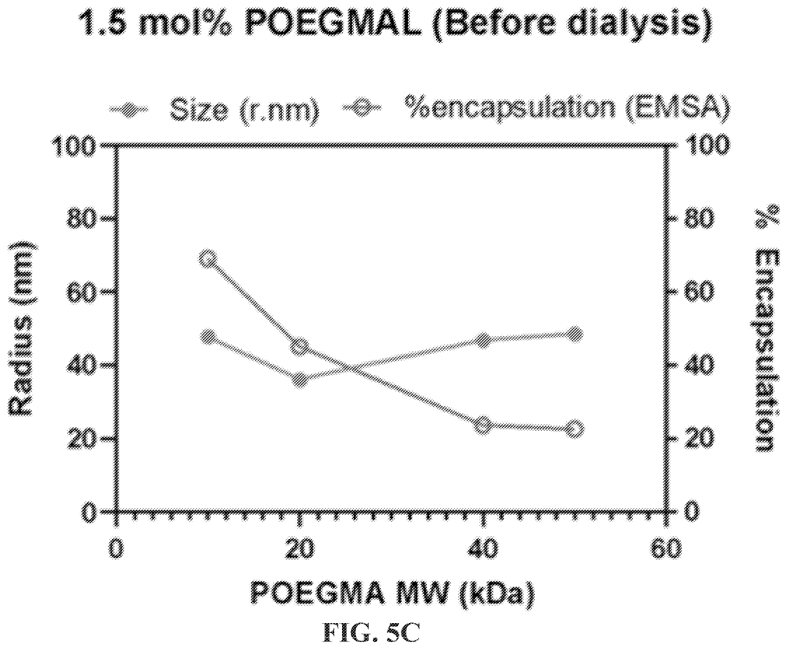

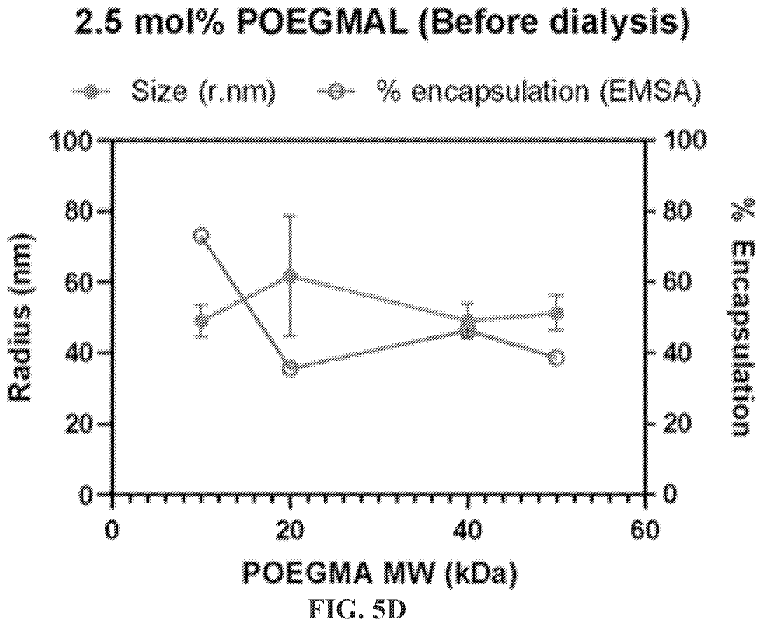

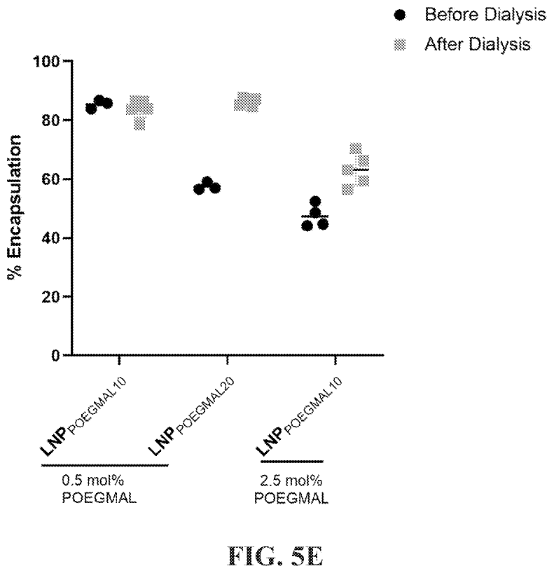

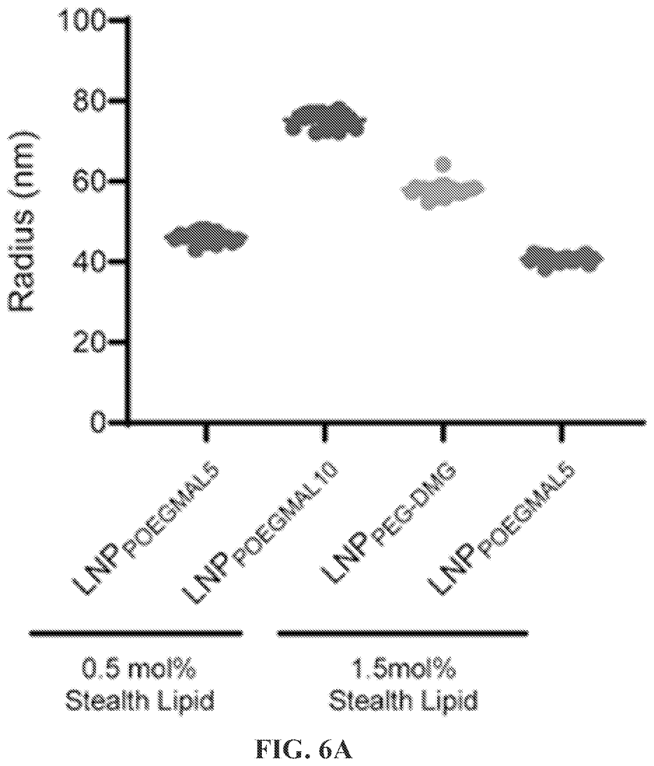

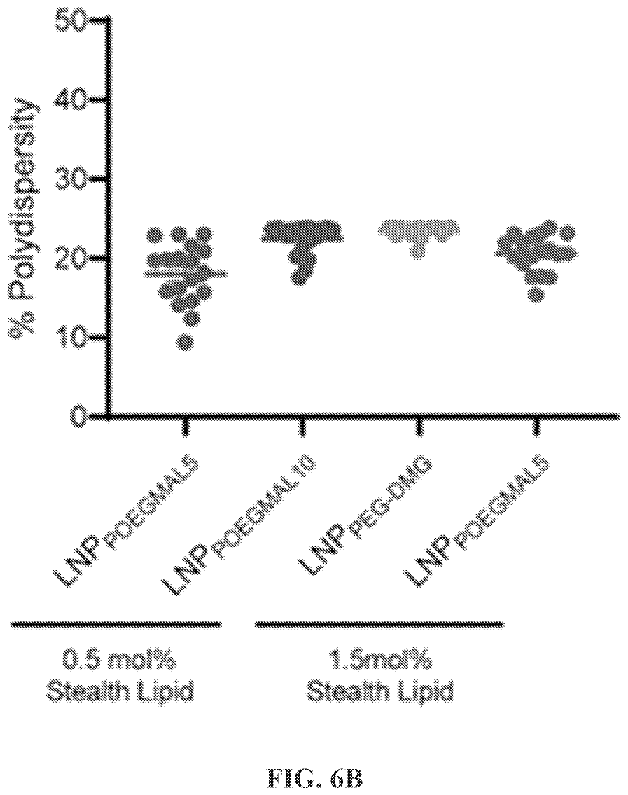

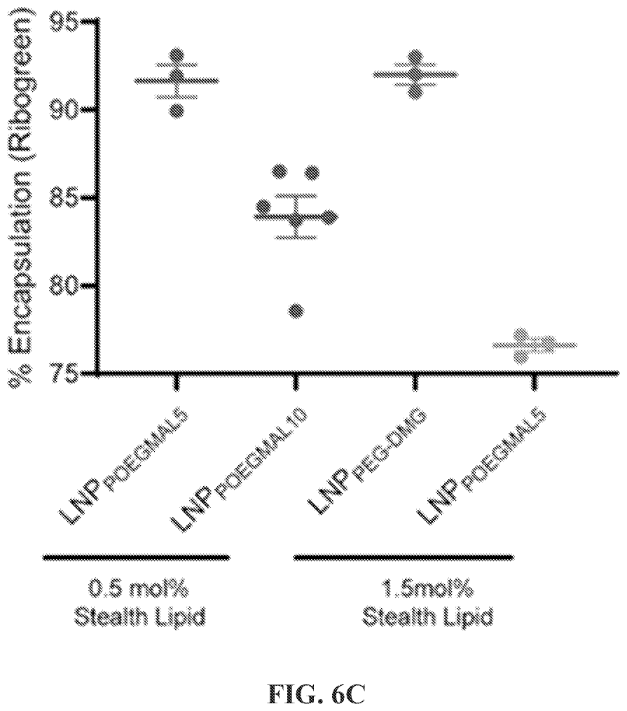

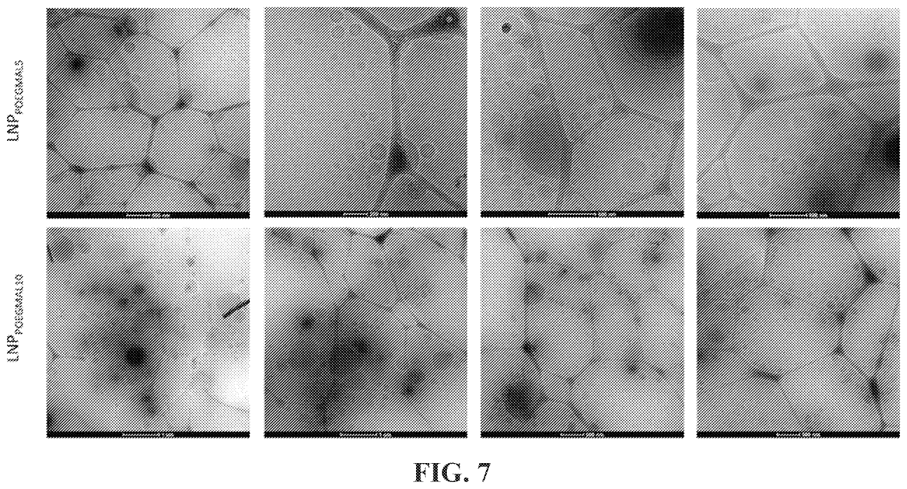

To obtain an improved encapsulation efficiency (EE), a one factor at a time (OFAT) study was designed to analyze the mol fraction of POEGMAL and SM-102 that could stably form nanoparticles while maximizing the EE. Mol % of the POEGMAL₁₀₋₅₀ and SM-102 was varied and the mRNA encapsulation was measured before dialysis with EMSA. As can be seen from FIG. 5A, POEGMAL₁₀ and POEGMAL₂₀ displayed an appreciable loading of ≥75%, which was set as a threshold to down select the LNP candidates. Changing the SM-102 mol % affected the EE. The relation between EE and nanoparticle size distribution are depicted in FIG. 5B, FIG. 5C, and FIG. 5D. All POEGMAL formed stable monodispersed nanoparticles at all mol % irrespective of their EE. The selected candidate (circled in FIG. 5A) was dialyzed against PBS and was characterized with DLS and Ribogreen assay. Although all selected candidates formed stable nanoparticles, LNPs of POEGMAL₁₀ at 0.5 mol % showed ˜2.5-fold increase in size and this formulation showed stable and reproducible EE>85% (FIG. 5E). Motivated by this fact, an OFAT study was performed with POEGMA₅ by varying mol % to assess whether further lowering of POEMAL's MW increases the EE. All formulations exhibited stable nanoparticles with below 100 nm radius and <30% polydispersity. EE was >90% before dialysis at all mol %, but 0.5 mol % was found to exhibit the highest EE after dialysis (FIG. 6A and FIG. 6B). Taken together, both POEGMAL₅ and POEGMAL₁₀ at 0.5 mol % formed stable LNPs that can retain more than 85% mRNA even after dialysis. So, it was decided to image these two formulations by cryo-TEM. As can be seen from FIG. 7, both formed spherical nanoparticles.

### Example 7

Encapsulation of a Therapeutically Relevant mRNA

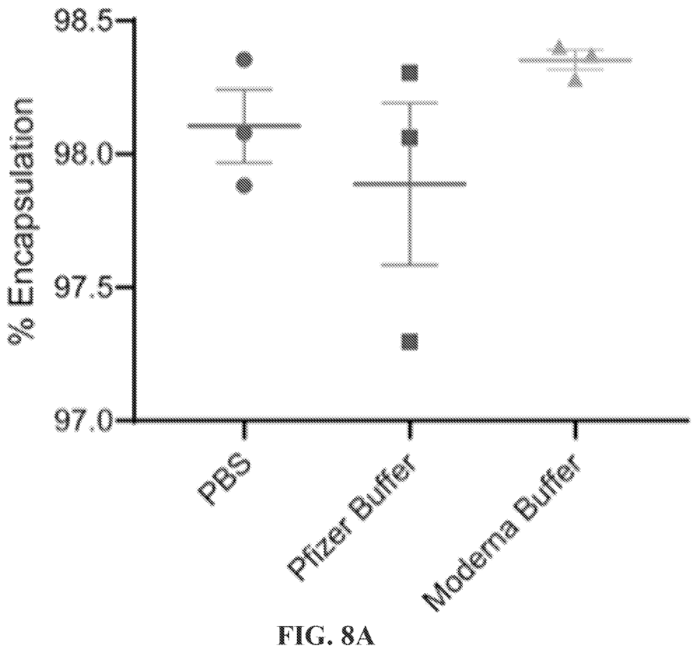

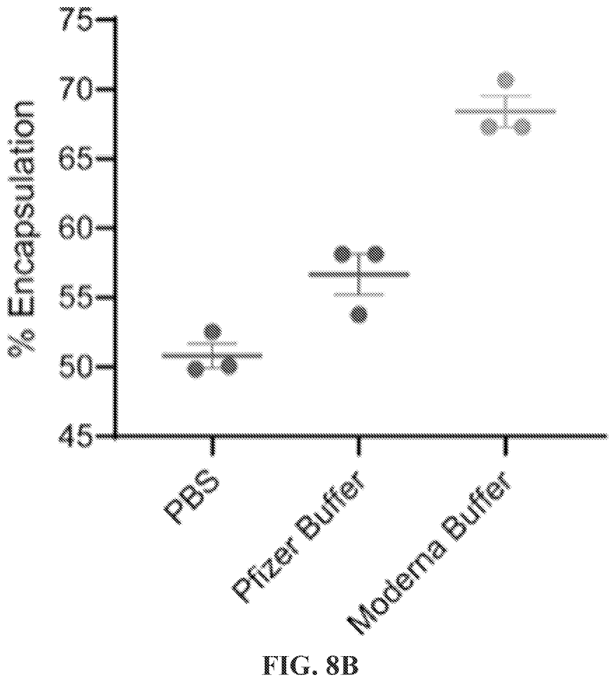

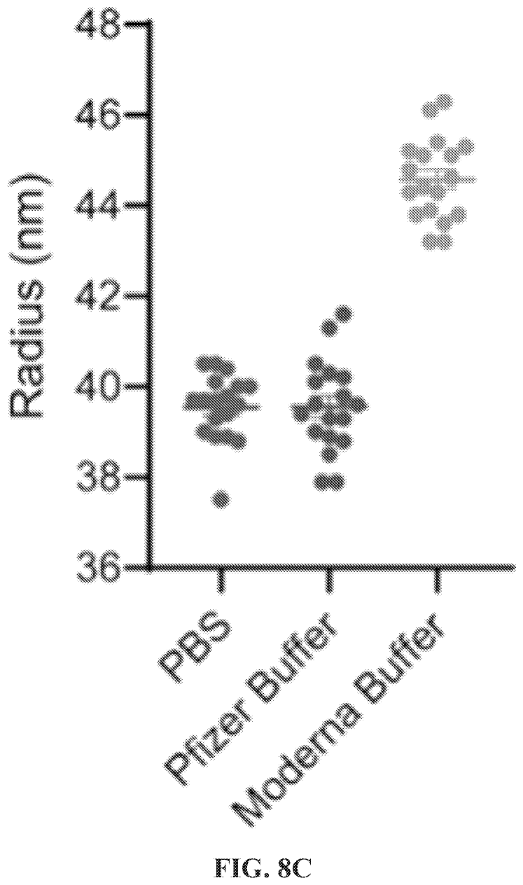

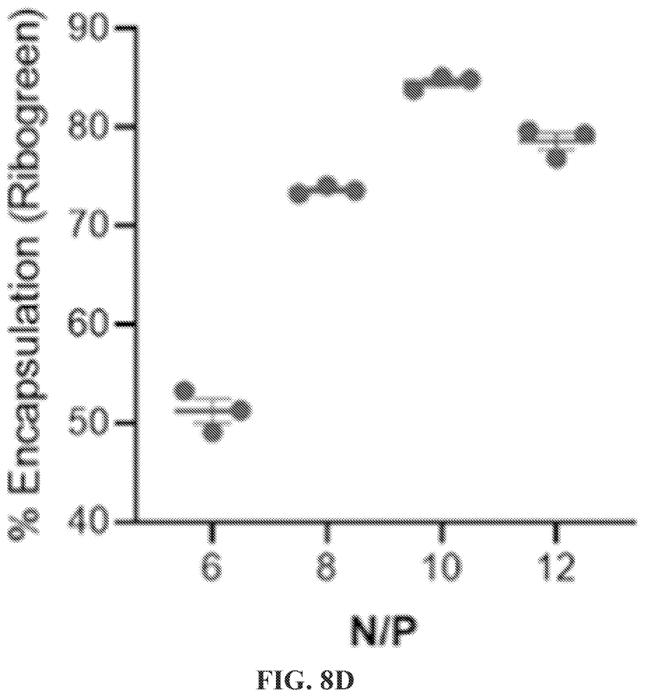

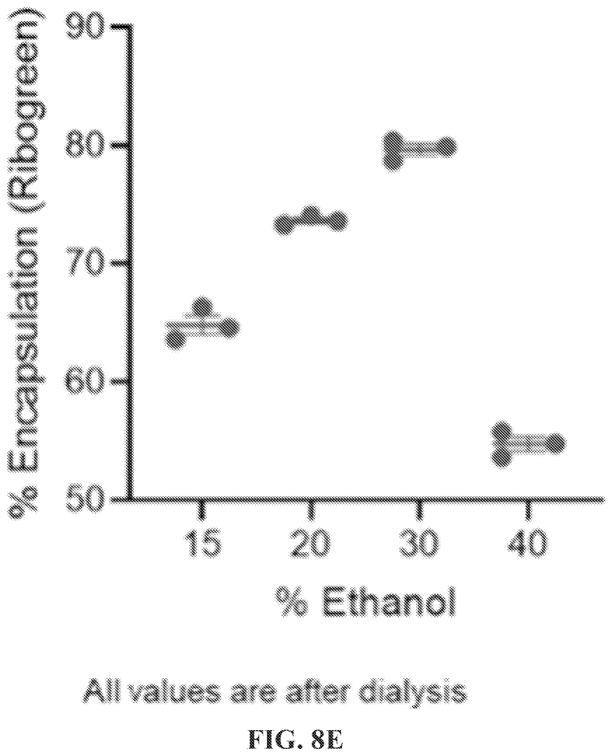

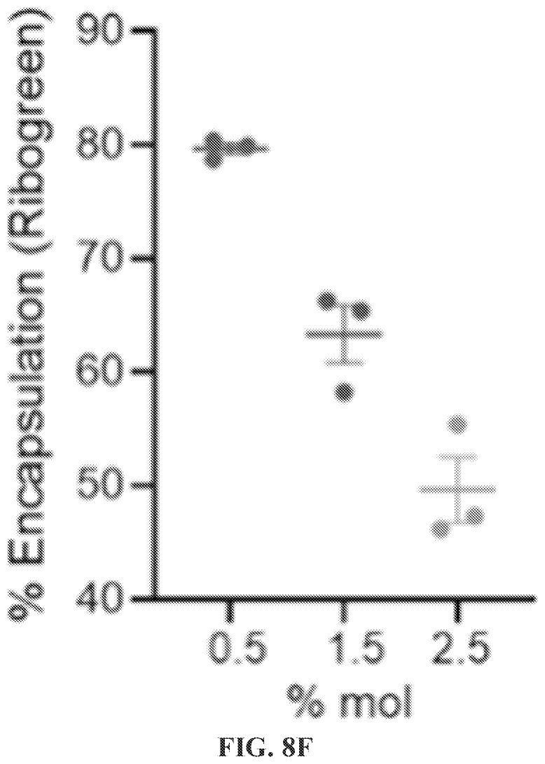

Next, the LNP system was probed to encapsulate therapeutically relevant SARS COV-2 spike protein mRNA. mRNA encoding the full-length SARS-CoV-2 spike glycoprotein was accessed from WHO International “Non-proprietary Names Programme”. This mRNA is supposedly used in Pfizer's COVID19 vaccine. Parameters for in-vitro transcription by T7 RNA polymerase were used to increase the mRNA yield and minimize formation of by-products (long transcripts and dsRNA). The formulation that was used is tailored for luciferase mRNA −0.5 mol % POEGMAL_{5&10}, N:P 8:1, 20% ethanol, dialyzed against PBS. The EE after dialysis was compromised with LNP_{POEGMAL5} exhibiting only ˜30% EE. This is not completely unexpected for three reasons: (i) the size of the SARS COV-2 mRNA is 2-fold larger than luciferase mRNA; (ii) the dialysis buffer is not optimal and (iii) the preparation method, lipid and N:P ratio may not be effective. All these challenges were probed with LNP_{POEGMAL10} (FIG. 8). First, the role of dialysis buffer was assessed (FIG. 8A and FIG. 8B). As the formulation is a biosimilar of Moderna, it showed improved EE (˜67%) in the buffer that is tailor made for Moderna LNP vaccine (FIG. 8B). Also, the sizes of LNP remained unchanged (FIG. 8C). Therefore, Moderna buffer was used for subsequent analysis. Second, the EE was assessed in a range of N:P and ethanol concentrations. It can be seen that N:P 10:1 (FIG. 8D) and 30% ethanol (FIG. 8E) are useful for encapsulating the mRNA, pushing the EE to more than 80% after dialysis. Third, a similar OFAT study was designed and found that 0.5 mol % is still the most effective lipid concentration for POEGMAL₁₀ (FIG. 8F) The surface potential that may increase at higher N:P ratio and can contribute to elevated toxicities of the LNPs was also assessed. Interestingly, the surface charge did not increase significantly, indicating at higher N:P, the mRNA content of the LNPs is increasing and neutralizing the charges. Surface charge of all the dialyzed formulations were below 10 mV. A zeta potential within this range is considered approximately neutral. Collectively, these data suggested that LNP_{POEGMAL10} can successfully encapsulate a model luciferase mRNA as well as a therapeutically relevant SARS COV-2 mRNA at high EE (˜85%).

### Example 8

### In Vitro Performance of LNPs

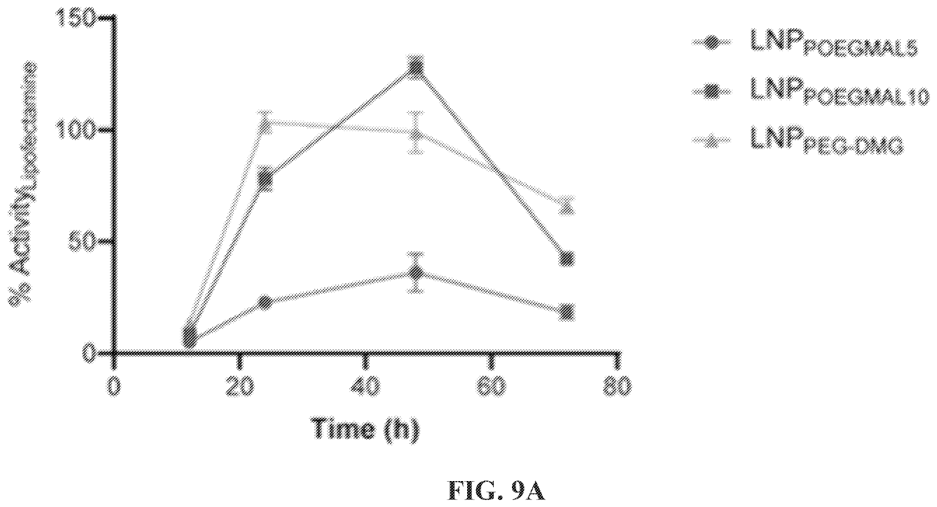

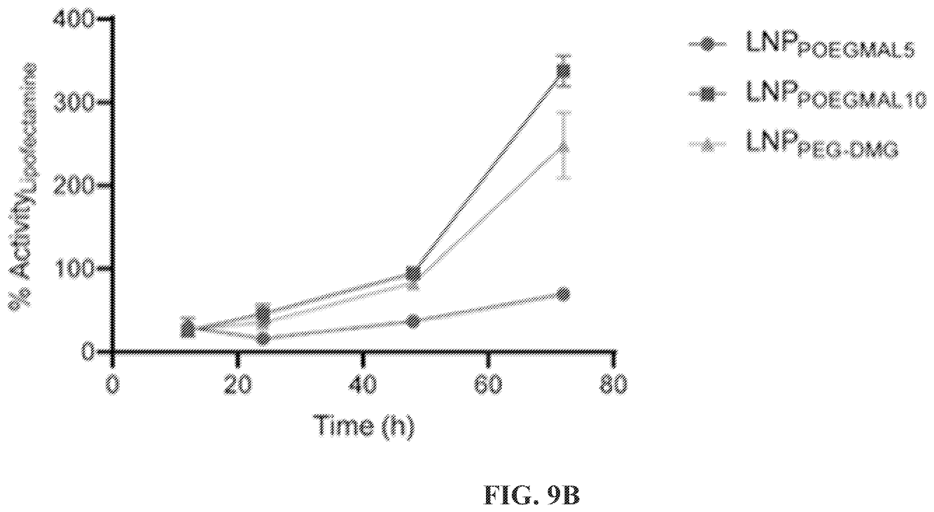

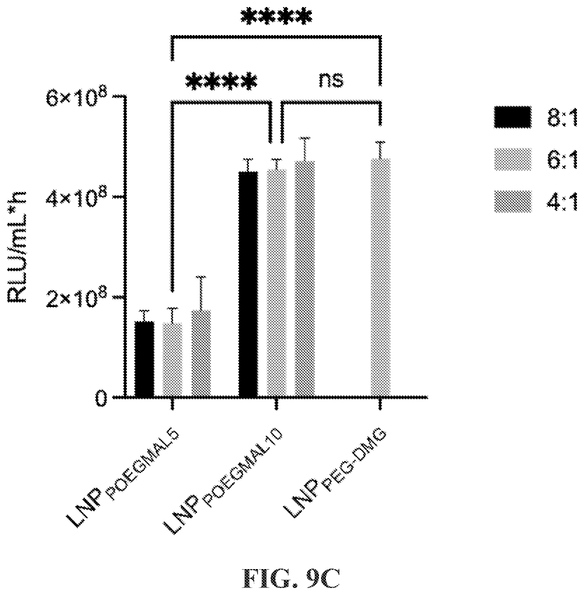

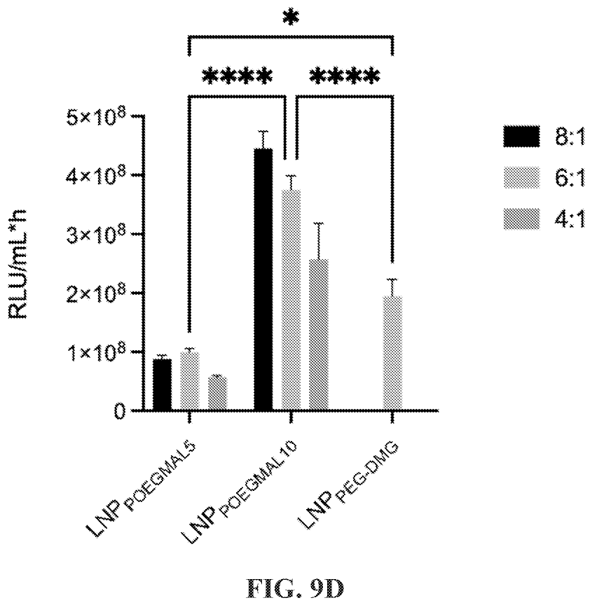

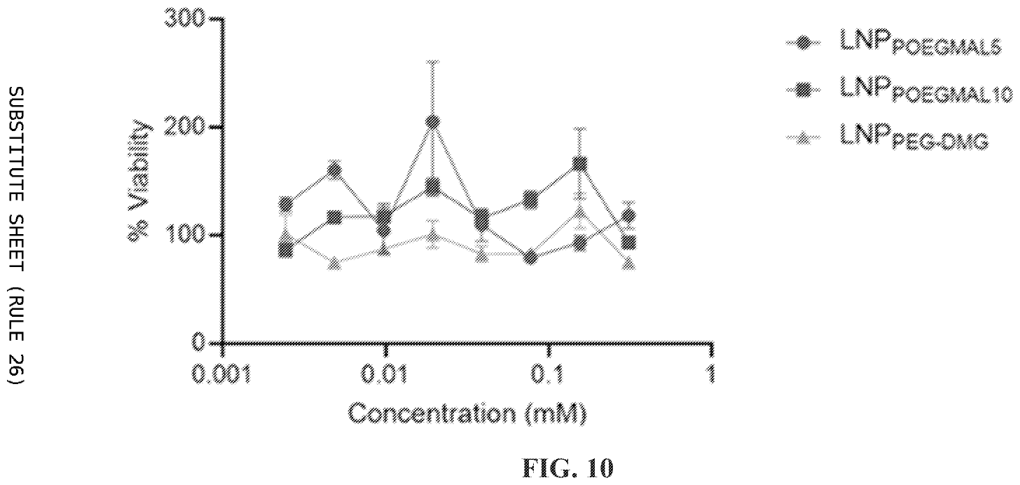

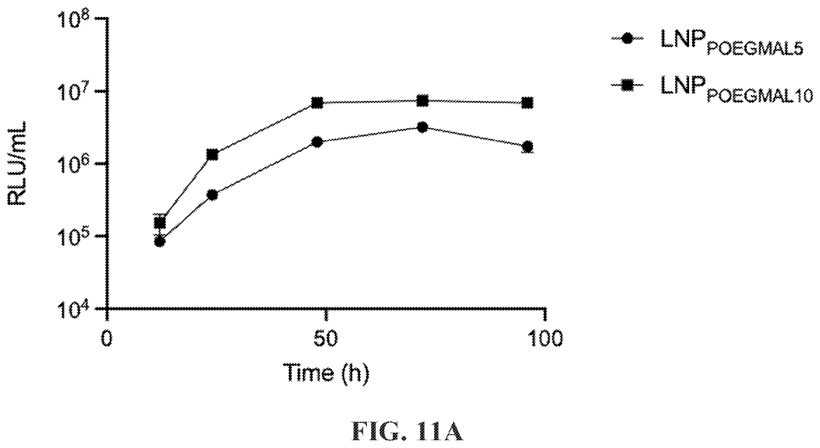

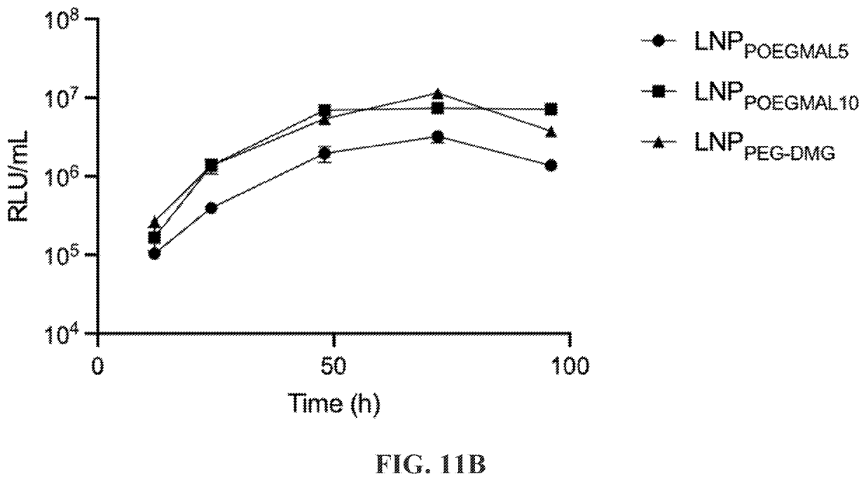

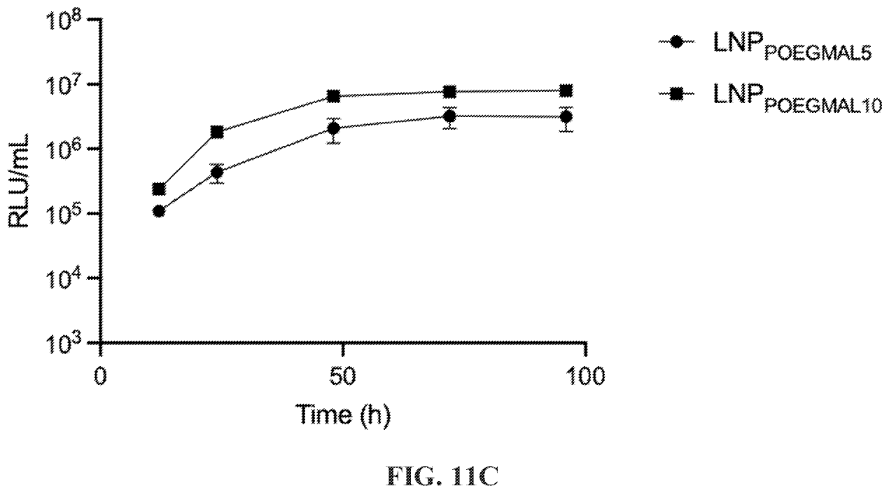

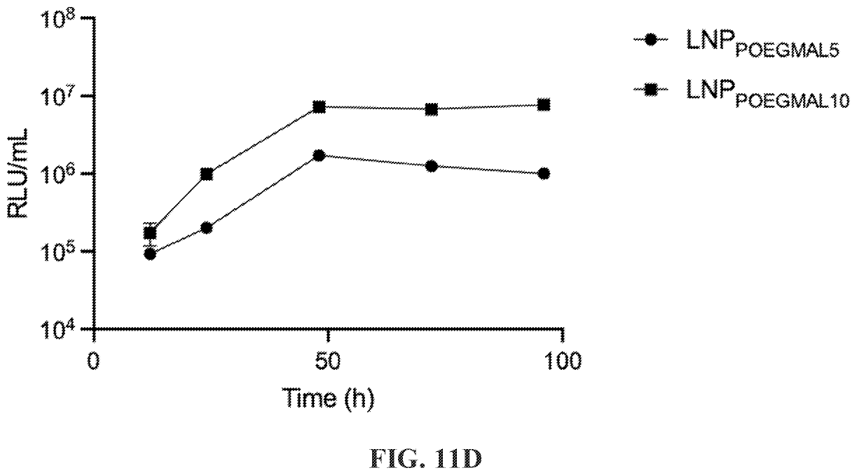

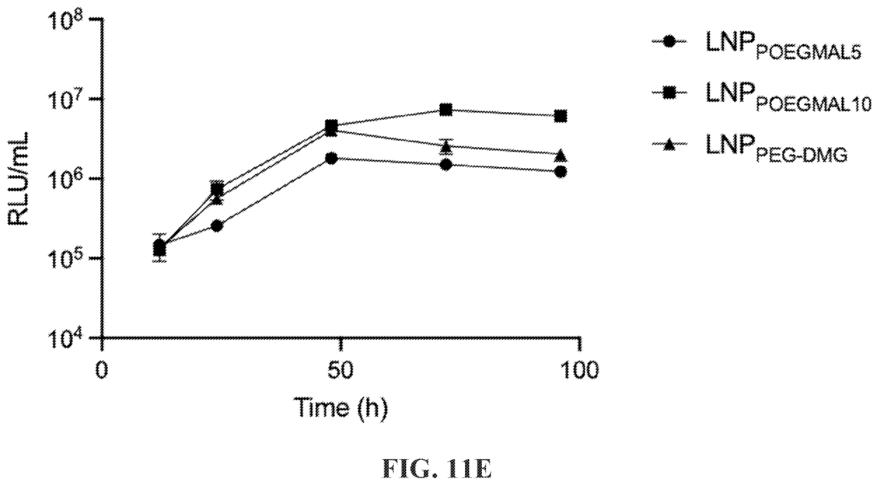

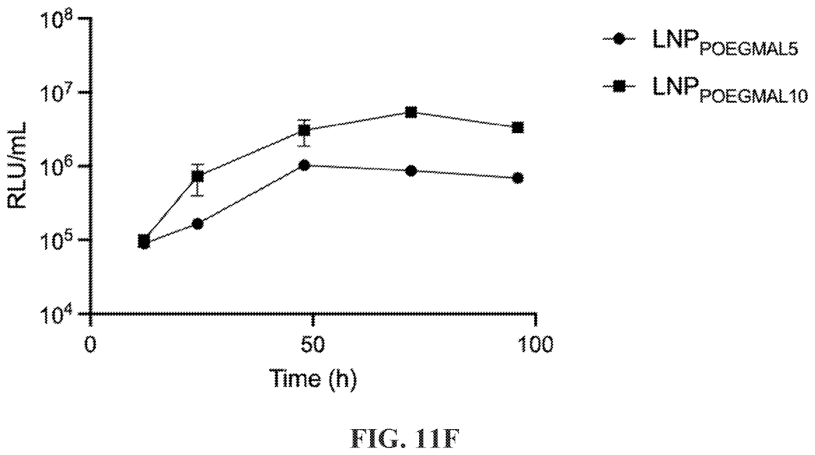

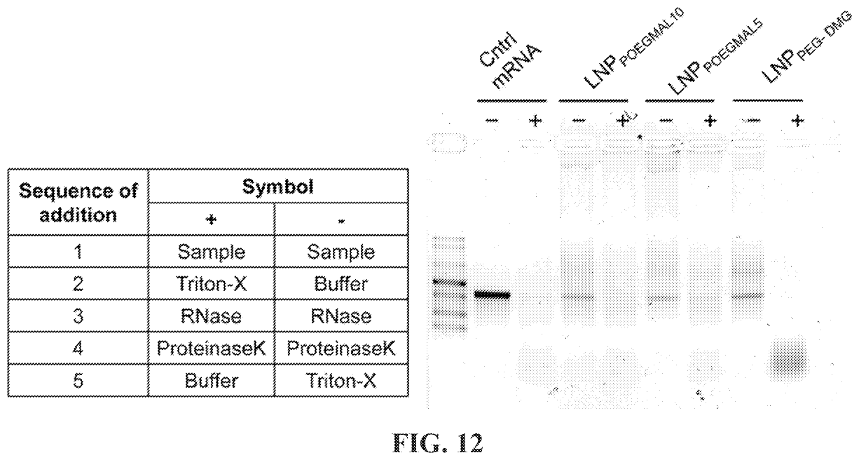

Finally, motivated by the fact that the present LNP system may serve as an alternative to deliver SARS COV-2 mRNA vaccine, the following were assessed: (i) toxicity in HEK293T cells; (ii) efficacy to express luciferase mRNA in HEK293T cell line over a period of time; and (iii) ability to protect the mRNA against RNase. Together these experiments serve as a model screening platform that is widely used in mRNA vaccine research to demonstrate potential in vivo utility. Moreover, the performance of the LNPs was benchmarked with Moderna biosimilar LNP_{PEG-DMG} and/or Lipofectamine 2000. The toxicity of the LNPs was assessed against HEK293T cells with LDH assay. Even after 48 h of continuous treatment, all the LNPs showed minimal toxicity (FIG. 10). To assess the luciferase expression, HEK293T cells were treated with various LNPs at N:P ratio of 4:1-8:1 and mRNA amount 300-500 ng. Summarized in FIG. 11A, FIG. 11B, FIG. 11C, FIG. 11D, FIG. 11E, and FIG. 11F, both LNP_{POEGMAL5} and LNP_{POEGMAL10} showed significant expression of the luciferase over 96 h. LNP_{POEGMAL10} WAS found to be better than LNP_{POEGMAL5}. At N:P—8:1 for LNP_{POEGMAL10} and 6:1 for LNPPEG-DMG—both formulations outperformed Lipofectamine 2000 (FIG. 9A and FIG. 9B). The difference in expression becomes more prominent at 300 ng of mRNA where LNP_{POEGMAL10} showed more than 300% luciferase activity than lipofectamine 2000 and outperformed the Moderna biosimilar LNP_{PEG-DMG}. The luciferase expression was measured over a period of ˜3 days, and allowed the measurement of the area under the curve (AUC) using trapezoidal rule. As summarized in FIG. 9C and FIG. 9D, LNP_{POEGMAL10} Outperformed LNP_{POEGMAL5} at all N:P ratios. An equally important observation is that when higher amount of mRNA (500 ng) was used, both LNP_{POEGMAL10} and LNP_{PEG-DMG} showed similar AUC. However, at lower mRNA amount (300 ng), LNP_{POEGMAL10} outperformed the Moderna biosimilar LNP_{PEG-DMG} with ˜2-fold higher AUC at identical N:P 6:1, and at the N:P 8:1 LNP_{POEGMAL10} showed 2.3-fold higher AUC than LNP_{PEG-DMG}. Finally, the efficacy of the LNPs to protect the mRNA cargo against RNase was investigated. This is an important parameter that dictates successful preclinical and clinical translation of LNP-mRNA vaccine. mRNA can be easily digested by extracellular RNase that results in poor in vivo performance. An in-house assay where the LNP-mRNA vaccine candidate was incubated with high concentration of RNase. An LNP rupturing agent was added before or after adding the RNase. After the incubation time the excess RNase was quenched with proteinaseK. As summarized in FIG. 12, all the candidate LNPs are able to give significant protection against RNase. The control free mRNA and the groups where Triton-X are added before adding the RNase shows complete degradation of the mRNA. Collectively, these data suggest POEGMAlyted LNP can serve as mRNA vaccine delivery platform and has high potential for successful clinical translation.

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A lipid nanoparticle comprising: an ionizable lipid; a phospholipid; a sterol; a poly[oligo (ethylene glycol) ether methacrylate] (POEGMA)-lipid conjugate at less than 10 mol %, wherein the POEGMA has a number average molecular weight of less than 100 kDa; and a therapeutic.

Clause 2. The lipid nanoparticle of clause 1, wherein the POEGMA has a poly (methyl methacrylate) backbone and a plurality of side chains covalently attached to the backbone, each side chain comprising 2 to 9 monomers of ethylene glycol (EG) repeated in tandem

Clause 3. The lipid nanoparticle of clause 1 or 2, wherein the lipid nanoparticle has a reduced immune response relative to a lipid nanoparticle including polyethylene glycol (PEG).

Clause 4. The lipid nanoparticle of any one of clauses 1-3, wherein the lipid nanoparticle is not reactive with pre-existing anti-PEG antibodies in a subject.

Clause 5. The lipid nanoparticle of any one of clauses 1-4, wherein the lipid of the POEGMA-lipid conjugate comprises a C₂₋₄₀ hydrocarbon chain.

Clause 6. The lipid nanoparticle of any one of clauses 1-5, wherein the lipid of the POEGMA-lipid conjugate is conjugated to the POEGMA through a triazole, an amide, an ester, an ether, or a hydrocarbon linker.

Clause 7. The lipid nanoparticle of any one of clauses 1-6, wherein the POEGMA has a number average molecular weight of about 1 kDa to about 50 kDa.

Clause 8. The lipid nanoparticle of any one of clauses 1-7, wherein the lipid nanoparticle comprises the POEGMA-lipid conjugate at about 0.1 mol % to about 10 mol %.

Clause 9. The lipid nanoparticle of any one of clauses 1-8, wherein the ionizable lipid comprises (heptadecan-9-yl 8-((2-hydroxyethyl) (6-oxO)-6-(undecyloxy) hexyl) amino) octanoate) (SM-102), 3,6-bis({4-[bis(2-hydroxydodecyl)amino]butyl})piperazine-2,5-dione (cKK-E12), 1-Linoleoyl-2-linoleyloxy-3-dmiethylaminopropane (DLin-2-DMAP), 1,2-Dilinoleylcarbanioyloxy-3-dimethylaniinopropane (DLin-C-DAP), 1,2-Dilmoleoyl-3-dimethylammopropane (DLm-DAP), 1,2-Dilinoleyloxy-N,N-dimethylaminopropane (DLin-DMA), 2,2-Dilinoleyl-4-dimethy laminomethy 1-[1,3]-dioxolane (DLin-K-DMA), 2,2-dilmoleyl-4-(2-dimethylaiiimoethyl)-[1,3]-dioxolane (DLin-KC2-DMA), (6Z,9Z,28Z,3 1Z)-heptatriaeonta-6,9,28,3 1-tetraen-19-yl 4-(dimethylamino) butanoate (DLin-MC3-DMA), 1,2-dioieoyl-3-dimethylammonium propane (DODAP), N,N-dimethyl-(2,3-dioleyloxy) propylamine (DODMA), dioctadecylamidoglycyocarboxyspermine (DOGS), spermine cholesterylcarbamate (GL-67), bis-guanidinium-spermidine-cholesterol (BGTC), 3b-(N-(N^{A}N′-dimethylammoethanej-carbam oxl cholesterol (DC-Chol), N-t-butyl-N′-tetradecylamino-propionamidine (diC14-amidine), dimethyldioctadecylammoniumbromide (DDAB), N-(1,2-dimyristyloxyprop-3-yl)-N,N-dimethyl-N-hydroxyethyl ammonium bromide (DMR1E), N,N-dioleyl-N,N-dimethylammonium chloride (DODAC), dioleyloxypropyl-3-dimethyl hydroxy ethyl ammonium bromide (DORIE), N-(1-(2,3-dioleyloxy3) propyl)-N-2-(spenninecarboxamido) ethyl)-N,N-dimethylamrnonium trifluoracetate (DOSPA), 2-dioleoy trimethyl ammonium propane chloride (DOTAP), N-(1-(2 3-dioleyloxy) propyl)-N N,N-trimethylammonium chloride (DOTMA), aminopropyl-dimethyl-bis(dodecyloxy)-propanaminiumbromide (GAP-DLRIE), 1,2-dioleoyl-sn-3-phosphoethanolamine (DOPE), (4-Hydroxybutyl)azanediyl]di(hexane-6,1-diyl)bis(2-hexyldecanoate) (ALC-0315), or a combination thereof.

Clause 10. The lipid nanoparticle of any one of clauses 1-9, wherein the ionizable lipid comprises (heptadecan-9-yl 8-((2-hydroxyethyl)(6-oxO-6-(undecyloxy) hexyl) amino) octanoate) (SM-102).

Clause 11. The lipid nanoparticle of any one of clauses 1-10, wherein the lipid nanoparticle comprises the ionizable lipid at about 20 mol % to about 65 mol %.

Clause 12. The lipid nanoparticle of any one of clauses 1-11, wherein the phospholipid comprises distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanol amine (D SPE), monomethyl-phosphatidylethanolamine, dimethyl-phosphatidylethanolamine, 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilinoleoyl-sn-glycero-3-phosphocholine (DLPC), 1,2-dimyristoyl-sn-glycero-3-phosphocholine (DMPC), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or a combination thereof.

Clause 13. The lipid nanoparticle of any one of clauses 1-12, wherein the lipid nanoparticle comprises the phospholipid at about 5 mol % to about 25 mol %.

Clause 14. The lipid nanoparticle of any one of clauses 1-13, wherein the sterol comprises cholesterol, campesterol, antrosterol, desmosterol, nicasterol, stigmasterol, sitosterol, oxysterol, C₄₋₁₀ sterol, ergosterol, cholest-4-en-3-one, or a combination thereof.

Clause 15. The lipid nanoparticle of any one of clauses 1-14, wherein the lipid nanoparticle comprises the sterol at about 10 mol % to about 50 mol %.

Clause 16. The lipid nanoparticle of any one of clauses 1-15, wherein the therapeutic is a nucleic acid comprising siRNA, miRNA, antisense oligonucleotides, shRNA, mRNA, tRNA, IRNA, CircRNA, DNA, or a combination thereof.

Clause 17. The lipid nanoparticle of clause 16, wherein the nucleic acid comprises siRNA, mRNA, or a combination thereof.

Clause 18. A lipid nanoparticle comprising. (heptadecan-9-yl 8-((2-hydroxyethyl)(6-oxO-6-(undecyloxy) hexyl) amino) octanoate) (SM-102); DSPC; cholesterol; a poly[oligo(ethylene glycol) ether methacrylate] (POEGMA)-lipid conjugate at about 0.25 mol % to about 3 mol %, wherein the POEGMA has a number average molecular weight of about 1 kDa to about 50 kDa; and an mRNA.

Clause 19. The lipid nanoparticle of any one of clauses 1-18, wherein the lipid nanoparticle has an N: P ratio of about 4:1 to about 16:1.

Clause 20. The lipid nanoparticle of any one of clauses 1-19, wherein the lipid nanoparticle has a diameter of about 30 nm to about 300 nm.

Clause 21. The lipid nanoparticle of any one of clauses 1-20, wherein the lipid nanoparticle has a therapeutic encapsulation efficiency of greater than or equal to 75% as measured by fluorescence.

Clause 22. The lipid nanoparticle of any one of clauses 1-21, further comprising a targeting ligand.

Clause 23. A pharmaceutical composition comprising: one or more lipid nanoparticles according to any one of clauses 1-22; and a pharmaceutically acceptable excipient.

Clause 24. A method of treating a disease or a disorder in a subject in need thereof, the method comprising administering to the subject an effective amount of one or more lipid nanoparticles according to any one of clauses 1-22, optionally in combination with a pharmaceutically acceptable excipient.

Clause 25. The method of clause 24, wherein the one or more lipid nanoparticles has a reduced immune response relative to a lipid nanoparticle including polyethylene glycol (PEG); is not reactive with pre-existing anti-PEG antibodies in the subject, or a combination thereof.

Clause 26. The method of clause 24 or 25, wherein the disease or the disorder is an infectious disease, Huntington's disease, muscular dystrophy, an autoimmune disease, or cancer.

Clause 27. A method of delivering a therapeutic to a cell, the method comprising contacting the cell with one or more lipid nanoparticles according to any one of clauses 1-22,whereby the therapeutic is delivered to the cell.

### Sequences

Cypridina luciferase mRNA:
SEQ ID NO: 1
GGGAGACCCAAGCUUGGUACCGAGCUCGGAUCCGCCACCAUGAAGACCUUAAUUCUUGCCGUUGCAUUAGUCUACUGCG
CCACUGUUCAUUGCCAGGACUGUCCUUACGAACCUGAUCCACCAAACACAGUUCCAACUUCCUGUGAAGCUAAAGAAGG
AGAAUGUAUUGAUAGCAGCUGUGGCACCUGCACGAGAGACAUACUAUCAGAUGGACUGUGUGAAAAUAAACCAGGAAAA
ACAUGUUGCCGAAUGUGUCAGUAUGUAAUUGAAUGCAGAGUAGAGGCCGCAGGAUGGUUUAGAACAUUCUAUGGAAAGA
GAUUCCAGUUCCAGGAACCUGGUACAUACGUGUUGGGUCAAGGAACCAAGGGCGGCGACUGGAAGGUGUCCAUCACCCU
GGAGAACCUGGAUGGAACCAAGGGGGCUGUGCUGACCAAGACAAGACUGGAAGUGGCUGGAGACAUCAUUGACAUCGCU
CAAGCUACUGAGAAUCCCAUCACUGUAAACGGUGGAGCUGACCCUAUCAUCGCCAACCCGUACACCAUCGGCGAGGUCA
CCAUCGCUGUUGUUGAGAUGCCAGGCUUCAACAUCACCGUCAUUGAGUUCUUCAAACUGAUCGUGAUCGACAUCCUCGG
AGGAAGAUCUGUAAGAAUCGCCCCAGACACAGCAAACAAAGGAAUGAUCUCUGGCCUCUGUGGAGAUCUUAAAAUGAUG
GAAGAUACAGACUUCACUUCAGAUCCAGAACAACUCGCUAUUCAGCCUAAGAUCAACCAGGAGUUUGACGGUUGUCCAC
UCUAUGGAAAUCCUGAUGACGUUGCAUACUGCAAAGGUCUUCUGGAGCCGUACAAGGACAGCUGCCGCAACCCCAUCAA
CUUCUACUACUACACCAUCUCCUGCGCCUUCGCCCGCUGUAUGGGUGGAGACGAGCGAGCCUCACACGUGCUGCUUGAC
UACAGGGAGACGUGCGCUGCUCCCGAAACUAGAGGAACCUGCGUUUUGUCUGGACAUACUUUCUACGAUACAUUUGACA
AAGCAAGAUACCAAUUCCAGGGUCCCUGCAAGGAGAUUCUUAUGGCCGCCGACUGUUUCUGGAACACUUGGGAUGUGAA
GGUUUCACACAGGAAUGUUGACUCUUACACUGAAGUAGAGAAAGUACGAAUCAGGAAACAAUCGACUGUAGUAGAACUC
AUUGUUGAUGGAAAACAGAUUCUGGUUGGAGGAGAAGCCGUGUCCGUCCCGUACAGCUCUCAGAACACUUCCAUCUACU
GGCAAGAUGGUGACAUACUGACUACAGCCAUCCUACCUGAAGCUCUGGUGGUCAAGUUCAACUUCAAGCAACUGCUCGU
CGUACAUAUUAGAGAUCCAUUCGAUGGUAAGACUUGCGGUAUUUGCGGUAACUACAACCAGGAUUUCAGUGAUGAUUCU
UUUGAUGCUGAAGGAGCCUGUGAUCUGACCCCCAACCCACCGGGAUGCACCGAAGAACAGAAACCUGAAGCUGAACGAC
UCUGCAAUAGUCUCUUCGCCGGUCAAAGUGAUCUUGAUCAGAAAUGUAACGUGUGCCACAAGCCUGACCGUGUCGAACG
AUGCAUGUACGAGUAUUGCCUGAGGGGACAACAGGGUUUCUGUGACCACGCAUGGGAGUUCAAGAAAGAAUGCUACAUA
AAGCAUGGAGACACCCUAGAAGUACCAGAUGAAUGCAAAUAGGCGGCCGCAAUAAAAUAUCUUUAUUUUCAUUACAUCU
GUGUGUUGGUUUUUUGUGUGUCUAG(A)₂₀₀₋₃₀₀
SARS-COV2 spike protein mRNA:
SEQ ID NO: 2
AGGAAUAAACUAGUAUUCUUCUGGUCCCCACAGACUCAGAGAGAACCCGCCACCAUGUUCGUGUUCCUGGUGCUGCUGC
CUCUGGUGUCCAGCCAGUGUGUGAACCUGACCACCAGAACACAGCUGCCUCCAGCCUACACCAACAGCUUUACCAGAGG
CGUGUACUACCCCGACAAGGUGUUCAGAUCCAGCGUGCUGCACUCUACCCAGGACCUGUUCCUGCCUUUCUUCAGCAAC
GUGACCUGGUUCCACGCCAUCCACGUGUCCGGCACCAAUGGCACCAAGAGAUUCGACAACCCCGUGCUGCCCUUCAACG
ACGGGGUGUACUUUGCCAGCACCGAGAAGUCCAACAUCAUCAGAGGCUGGAUCUUCGGCACCACACUGGACAGCAAGAC
CCAGAGCCUGCUGAUCGUGAACAACGCCACCAACGUGGUCAUCAAAGUGUGCGAGUUCCAGUUCUGCAACGACCCCUUC
CUGGGCGUCUACUACCACAAGAACAACAAGAGCUGGAUGGAAAGCGAGUUCCGGGUGUACAGCAGCGCCAACAACUGCA
CCUUCGAGUACGUGUCCCAGCCUUUCCUGAUGGACCUGGAAGGCAAGCAGGGCAACUUCAAGAACCUGCGCGAGUUCGU
GUUUAAGAACAUCGACGGCUACUUCAAGAUCUACAGCAAGCACACCCCUAUCAACCUCGUGCGGGAUCUGCCUCAGGGC
UUCUCUGCUCUGGAACCCCUGGUGGAUCUGCCCAUCGGCAUCAACAUCACCCGGUUUCAGACACUGCUGGCCCUGCACA
GAAGCUACCUGACACCUGGCGAUAGCAGCAGCGGAUGGACAGCUGGUGCCGCCGCUUACUAUGUGGGCUACCUGCAGCC
UAGAACCUUCCUGCUGAAGUACAACGAGAACGGCACCAUCACCGACGCCGUGGAUUGUGCUCUGGAUCCUCUGAGCGAG
ACAAAGUGCACCCUGAAGUCCUUCACCGUGGAAAAGGGCAUCUACCAGACCAGCAACUUCCGGGUGCAGCCCACCGAAU
CCAUCGUGCGGUUCCCCAAUAUCACCAAUCUGUGCCCCUUCGGCGAGGUGUUCAAUGCCACCAGAUUCGCCUCUGUGUA
CGCCUGGAACCGGAAGCGGAUCAGCAAUUGCGUGGCCGACUACUCCGUGCUGUACAACUCCGCCAGCUUCAGCACCUUC
AAGUGCUACGGCGUGUCCCCUACCAAGCUGAACGACCUGUGCUUCACAAACGUGUACGCCGACAGCUUCGUGAUCCGGG
GAGAUGAAGUGCGGCAGAUUGCCCCUGGACAGACAGGCAAGAUCGCCGACUACAACUACAAGCUGCCCGACGACUUCAC
CGGCUGUGUGAUUGCCUGGAACAGCAACAACCUGGACUCCAAAGUCGGCGGCAACUACAAUUACCUGUACCGGCUGUUC
CGGAAGUCCAAUCUGAAGCCCUUCGAGCGGGACAUCUCCACCGAGAUCUAUCAGGCCGGCAGCACCCCUUGUAACGGCG
UGGAAGGCUUCAACUGCUACUUCCCACUGCAGUCCUACGGCUUUCAGCCCACAAAUGGCGUGGGCUAUCAGCCCUACAG
AGUGGUGGUGCUGAGCUUCGAACUGCUGCAUGCCCCUGCCACAGUGUGCGGCCCUAAGAAAAGCACCAAUCUCGUGAAG
AACAAAUGCGUGAACUUCAACUUCAACGGCCUGACCGGCACCGGCGUGCUGACAGAGAGCAACAAGAAGUUCCUGCCAU
UCCAGCAGUUUGGCCGGGAUAUCGCCGAUACCACAGACGCCGUUAGAGAUCCCCAGACACUGGAAAUCCUGGACAUCAC
CCCUUGCAGCUUCGGCGGAGUGUCUGUGAUCACCCCUGGCACCAACACCAGCAAUCAGGUGGCAGUGCUGUACCAGGAC
GUGAACUGUACCGAAGUGCCCGUGGCCAUUCACGCCGAUCAGCUGACACCUACAUGGCGGGUGUACUCCACCGGCAGCA
AUGUGUUUCAGACCAGAGCCGGCUGUCUGAUCGGAGCCGAGCACGUGAACAAUAGCUACGAGUGCGACAUCCCCAUCGG
CGCUGGAAUCUGCGCCAGCUACCAGACACAGACAAACAGCCCUCGGAGAGCCAGAAGCGUGGCCAGCCAGAGCAUCAUU
GCCUACACAAUGUCUCUGGGCGCCGAGAACAGCGUGGCCUACUCCAACAACUCUAUCGCUAUCCCCACCAACUUCACCA
UCAGCGUGACCACAGAGAUCCUGCCUGUGUCCAUGACCAAGACCAGCGUGGACUGCACCAUGUACAUCUGCGGCGAUUC
CACCGAGUGCUCCAACCUGCUGCUGCAGUACGGCAGCUUCUGCACCCAGCUGAAUAGAGCCCUGACAGGGAUCGCCGUG
GAACAGGACAAGAACACCCAAGAGGUGUUCGCCCAAGUGAAGCAGAUCUACAAGACCCCUCCUAUCAAGGACUUCGGCG
GCUUCAAUUUCAGCCAGAUUCUGCCCGAUCCUAGCAAGCCCAGCAAGCGGAGCUUCAUCGAGGACCUGCUGUUCAACAA
AGUGACACUGGCCGACGCCGGCUUCAUCAAGCAGUAUGGCGAUUGUCUGGGCGACAUUGCCGCCAGGGAUCUGAUUUGC
GCCCAGAAGUUUAACGGACUGACAGUGCUGCCUCCUCUGCUGACCGAUGAGAUGAUCGCCCAGUACACAUCUGCCCUGC
UGGCCGGCACAAUCACAAGCGGCUGGACAUUUGGAGCAGGCGCCGCUCUGCAGAUCCCCUUUGCUAUGCAGAUGGCCUA
CCGGUUCAACGGCAUCGGAGUGACCCAGAAUGUGCUGUACGAGAACCAGAAGCUGAUCGCCAACCAGUUCAACAGCGCC
AUCGGCAAGAUCCAGGACAGCCUGAGCAGCACAGCAAGCGCCCUGGGAAAGCUGCAGGACGUGGUCAACCAGAAUGCCC
AGGCACUGAACACCCUGGUCAAGCAGCUGUCCUCCAACUUCGGCGCCAUCAGCUCUGUGCUGAACGAUAUCCUGAGCAG
ACUGGACCCUCCUGAGGCCGAGGUGCAGAUCGACAGACUGAUCACAGGCAGACUGCAGAGCCUCCAGACAUACGUGACC
CAGCAGCUGAUCAGAGCCGCCGAGAUUAGAGCCUCUGCCAAUCUGGCCGCCACCAAGAUGUCUGAGUGUGUGCUGGGCC
AGAGCAAGAGAGUGGACUUUUGOGGCAAGGGCUACCACCUGAUGAGCUUCCCUCAGUCUGCCCCUCACGGCGUGGUGUU
UCUGCACGUGACAUAUGUGCCCGCUCAAGAGAAGAAUUUCACCACCGCUCCAGCCAUCUGCCACGACGGCAAAGCCCAC
UUUCCUAGAGAAGGCGUGUUCGUGUCCAACGGCACCCAUUGGUUCGUGACACAGCGGAACUUCUACGAGCCCCAGAUCA
UCACCACCGACAACACCUUCGUGUCUGGCAACUGCGACGUCGUGAUCGGCAUUGUGAACAAUACCGUGUACGACCCUCU
GCAGCCCGAGCUGGACAGCUUCAAAGAGGAACUGGACAAGUACUUUAAGAACCACACAAGCCCCGACGUGGACCUGGGC
GAUAUCAGCGGAAUCAAUGCCAGCGUCGUGAACAUCCAGAAAGAGAUCGACCGGCUGAACGAGGUGGCCAAGAAUCUGA
ACGAGAGCCUGAUCGACCUGCAAGAACUGGGGAAGUACGAGCAGUACAUCAAGUGGCCCUGGUACAUCUGGCUGGGCUU
UAUCGCCGGACUGAUUGCCAUCGUGAUGGUCACAAUCAUGCUGUGUUGCAUGACCAGCUGCUGUAGCUGCCUGAAGGGC
UGUUGUAGCUGUGGCAGCUGCUGCAAGUUCGACGAGGACGAUUCUGAGCCCGUGCUGAAGGGCGUGAAACUGCACUACA
CAUGAUGACUCGAGCUGGUACUGCAUGCACGCAAUGCUAGCUGCCCCUUUCCCGUCCUGGGUACCCCGAGUCUCCCCCG
ACCUCGGGUCCCAGGUAUGCUCCCACCUCCACCUGCCCCACUCACCACCUCUGCUAGUUCCAGACACCUCCCAAGCACG
CAGCAAUGCAGCUCAAAACGCUUAGCCUAGCCACACCCCCACGGGAAACAGCAGUGAUUAACCUUUAGCAAUAAACGAA
AGUUUAACUAAGCUAUACUAACCCCAGGGUUGGUCAAUUUCGUGCCAGCCACACCCUGGAGCUAGCAAAAAAAAAAAAA
AAAAAAAAAAAAAAAAAGCAUAUGACUAAAAAAAAAAAAAAAAAAAAAAAAAAAAAAAAAAAAAAAAAAAAAAAAAAAA
AAAAAAAAAAAAAAAAAA

What is claimed is:

1. A lipid nanoparticle comprising:
an ionizable lipid;
a phospholipid;
a sterol;
a poly[oligo (ethylene glycol) ether methacrylate] (POEGMA)-lipid conjugate at less than 10 mol %, wherein the POEGMA has a number average molecular weight of less than 100 kDa; and
a therapeutic.

2. The lipid nanoparticle of claim 1, wherein the POEGMA has a poly (methyl methacrylate) backbone and a plurality of side chains covalently attached to the backbone, each side chain comprising 2 to 9 monomers of ethylene glycol (EG) repeated in tandem.

3. The lipid nanoparticle of claim 1, wherein the lipid nanoparticle has a reduced immune response relative to a lipid nanoparticle including polyethylene glycol (PEG).

4. The lipid nanoparticle of claim 1, wherein the lipid nanoparticle is not reactive with pre-existing anti-PEG antibodies in a subject.

5. The lipid nanoparticle of claim 1, wherein the lipid of the POEGMA-lipid conjugate comprises a C₂₋₄₀ hydrocarbon chain.

6. The lipid nanoparticle of claim 1, wherein the lipid of the POEGMA-lipid conjugate is conjugated to the POEGMA through a triazole, an amide, an ester, an ether, or a hydrocarbon linker.

7. The lipid nanoparticle of claim 1, wherein the POEGMA has a number average molecular weight of about 1 kDa to about 50 kDa.

8. The lipid nanoparticle of claim 1, wherein the lipid nanoparticle comprises the POEGMA-lipid conjugate at about 0.1 mol % to about 10 mol %.

9. The lipid nanoparticle of claim 1, wherein the ionizable lipid comprises (heptadecan-9-yl 8-((2-hydroxyethyl)(6-oxO-6-(undecyloxy) hexyl) amino) octanoate) (SM-102), 3,6-bis({4-[bis(2-hydroxydodecyl)amino]butyl})piperazine-2,5-dione (cKK-E12), 1-Linoleoyl-2-linoleyloxy-3-dmiethylaminopropane (DLin-2-DMAP), 1,2-Dilinoleylcarbanioyloxy-3-dimethylaniinopropane (DLin-C-DAP), 1,2-Dilmoleoyl-3-dimethylammopropane (DLm-DAP), 1,2-Dilinoleyloxy-N,N-dimethylaminopropane (DLin-DMA), 2,2-Dilinoleyl-4-dimethy laminomethy 1-[1,3]-dioxolane (DLin-K-DMA), 2,2-dilmoleyl-4-(2-dimethylaiiimoethyl)-[1,3]-dioxolane (DLin-KC2-DMA), (6Z,9Z,28Z,3 1Z)-heptatriaeonta-6,9,28,3 1-tetraen-19-yl 4-(dimethylamino) butanoate (DLin-MC3-DMA), 1,2-dioieoyl-3-dimethylammonium propane (DODAP), N,N-dimethyl-(2,3-dioleyloxy) propylamine (DODMA), dioctadecylamidoglycyocarboxyspermine (DOGS), spermine cholesterylcarbamate (GL-67), bis-guanidinium-spermidine-cholesterol (BGTC), 3b-(N-(N^{A}N′-dimethylammoethanej-carbam oxl cholesterol (DC-Chol), N-t-butyl-N′-tetradecylamino-propionamidine (diC14-amidine), dimethyldioctadecylammoniumbromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), dioleyloxypropyl-3-dimethyl hydroxy ethyl ammonium bromide (DORIE), N-(1-(2,3-dioleyloxy3) propyl)-N-2-(spenninecarboxamido) ethyl)-N,N-dimethylammonium trifluoracetate (DOSPA), 2-dioleoy trimethyl ammonium propane chloride (DOTAP), N-(1-(2 3-dioleyloxy)propyl)-N N,N-trimethylammonium chloride (DOTMA), aminopropyl-dimethyl-bis(dodecyloxy)-propanaminiumbromide (GAP-DLRIE), 1,2-dioleoyl-sn-3-phosphoethanolamine (DOPE), (4-Hydroxybutyl)azanediyl]di(hexane-6,1-diyl) bis(2-hexyldecanoate) (ALC-0315), or a combination thereof.

10. The lipid nanoparticle of claim 1, wherein the ionizable lipid comprises (heptadecan-9-yl 8-((2-hydroxyethyl)(6-oxO-6-(undecyloxy) hexyl) amino) octanoate) (SM-102).

11. The lipid nanoparticle of claim 1, wherein the lipid nanoparticle comprises the ionizable lipid at about 20 mol % to about 65 mol %.

12. The lipid nanoparticle of claim 1, wherein the phospholipid comprises distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanol amine (D SPE), monomethyl-phosphatidylethanolamine, dimethyl-phosphatidylethanolamine, 18-1-trans PE, 1-stearoyl-2-oleoyl-phosphatidyethanolamine (SOPE), hydrogenated soy phosphatidylcholine (HSPC), egg phosphatidylcholine (EPC), dioleoy lphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilinoleoyl-sn-glycero-3-phosphocholine (DLPC), 1,2-dimyristoyl-sn-glycero-3-phosphocholine (DMPC), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or a combination thereof.

13. The lipid nanoparticle of claim 1, wherein the lipid nanoparticle comprises the phospholipid at about 5 mol % to about 25 mol %.

14. The lipid nanoparticle of claim 1, wherein the sterol comprises cholesterol, campesterol, antrosterol, desmosterol, nicasterol, stigmasterol, sitosterol, oxysterol, C₄₋₁₀ sterol, ergosterol, cholest-4-en-3-one, or a combination thereof.

15. The lipid nanoparticle of claim 1, wherein the lipid nanoparticle comprises the sterol at about 10 mol % to about 50 mol %.

16. The lipid nanoparticle of claim 1, wherein the therapeutic is a nucleic acid comprising siRNA, miRNA, antisense oligonucleotides, shRNA, mRNA, tRNA, IRNA, CircRNA, DNA, or a combination thereof.

17. The lipid nanoparticle of claim 16, wherein the nucleic acid comprises siRNA, mRNA, or a combination thereof.

18. A lipid nanoparticle comprising:
(heptadecan-9-yl 8-((2-hydroxyethyl) (6-oxO-6-(undecyloxy) hexyl) amino) octanoate) (SM-102);
DSPC;
cholesterol;
a poly[oligo (ethylene glycol) ether methacrylate] (POEGMA)-lipid conjugate at about 0.25 mol % to about 3 mol %, wherein the POEGMA has a number average molecular weight of about 1 kDa to about 50 kDa; and
an mRNA.

19. The lipid nanoparticle of claim 1, wherein the lipid nanoparticle has an N:P ratio of about 4:1 to about 16:1.

20. The lipid nanoparticle of claim 1, wherein the lipid nanoparticle has a diameter of about 30 nm to about 300 nm.

21. The lipid nanoparticle of claim 1, wherein the lipid nanoparticle has a therapeutic encapsulation efficiency of greater than or equal to 75% as measured by fluorescence.

22. The lipid nanoparticle of claim 1, further comprising a targeting ligand.

23. A pharmaceutical composition comprising:
one or more lipid nanoparticles according to claim 1; and
a pharmaceutically acceptable excipient.

24. A method of treating a disease or a disorder in a subject in need thereof, the method comprising administering to the subject an effective amount of one or more lipid nanoparticles according to claim 1, optionally in combination with a pharmaceutically acceptable excipient.

25. The method of claim 24, wherein the one or more lipid nanoparticles has a reduced immune response relative to a lipid nanoparticle including polyethylene glycol (PEG); is not reactive with pre-existing anti-PEG antibodies in the subject, or a combination thereof.

26. The method of claim 24, wherein the disease or the disorder is an infectious disease, Huntington's disease, muscular dystrophy, an autoimmune disease, or cancer.

27. A method of delivering a therapeutic to a cell, the method comprising contacting the cell with one or more lipid nanoparticles according to claim 1, whereby the therapeutic is delivered to the cell.


SEQUENCE LISTING (2 sequences)

SEQ ID NO: 1 - RNA, 1764 nt, synthetic construct
  source 1..1764: mol_type = other RNA
  repeat_region 1764: rpt_unit_seq = 200 to 400
gggagacccaagcttggtaccgagctcggatccgccaccatgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggccgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcattgagttcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctggagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagataccaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaataggcggccgcaataaaatatctttattttcattacatctgtgtgttggttttttgtgtgtctaga

SEQ ID NO: 2 - RNA, 4284 nt, synthetic construct
  source 1..4284: mol_type = other RNA
aggaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa